(19) United States
(12) Patent Application Publication
(10) Pub. No.: US 2024/0374309 A1
(43) Pub. Date: Nov. 14, 2024

(54) ROBOTICALLY CONTROLLED LASER-ASSISTED HANDPIECE

(71) Applicants: Smith & Nephew, Inc., Memphis, TN (US); Smith & Nephew Asia Pacific Pte. Limited, SG (SG); Smith & Nephew Orthopaedics AG, Zug (CH)

(72) Inventors: Darren J. WILSON, Hull (GB); Brett J. BELL, Mendon, UT (US)

(21) Appl. No.: 18/662,181

(22) Filed: May 13, 2024

Related U.S. Application Data
(60) Provisional application No. 63/466,140, filed on May 12, 2023.

(51) Int. Cl. A61B 18/20; A61B 18/00; A61B 34/30; A61B 90/00

(52) U.S. Cl. CPC A61B 18/20; A61B 34/30; A61B 90/37; A61B 2018/00565; A61B 2018/00577; A61B 2090/378

(57) ABSTRACT

A robotically controlled laser resection device is disclosed. The device is configured in a handheld form factor. The device includes a treatment laser configured to generate a treatment laser beam, a laser scanner configured to direct the treatment laser beam, a water nozzle configured to emit a laminar waterjet, and an aperture configured to emit the treatment laser beam within the laminar waterjet. The components may be contained in a housing with electromagnetic shielding. A tracking marker is attached to the housing to allow navigation of the device.

### CROSS REFERENCE TO RELATED APPLICATIONS

The present application claims priority to U.S. Provisional Application No. 63/466,140, titled “ROBOTICALLY CONTROLLED LASER-ASSISTED HANDPIECE,” filed May 12, 2023, which is hereby incorporated by reference in its entirety.

### TECHNICAL FIELD

The present disclosure relates generally to methods, systems, and apparatuses related to surgical resection. More particularly, the present disclosure relates to a hand-held, semi-autonomous robotic resection tool, which the surgeon can move freely in space. The handpiece can offer intra-operative planning and depth control with functional laser cut geometries, and robotic assistance for a precise execution, and repeatability of movements. The robotically controlled, free-hand resection tool can integrate a portable ablation laser (e.g., a compact, diode pumped solid-state laser with an additional co-axially bundled aiming beam) or be tethered to an external laser.

### BACKGROUND

Bone tissue is an anisotropic, viscoelastic, hard, and brittle composite material. These characteristics can cause complications when performing a surgical resection for which the main requirements are high efficiency, high accuracy, and a low cutting temperature. In particular, the cutting efficiency of surgical instruments can affect the time of the entire surgical procedure, which can reduce patient throughput. Increasing cutting efficiency can often come at the expense of also increasing cutting temperature, which can reduce patient outcomes associated with thermal necrosis and cracking of the resected tissue that can lead to aseptic loosening of the implant.

Orthopedic oscillating saws are widely used for plane processing during joint replacement procedures with bone removal rates reported to be 5 to 7 mm³/s. However, a sagittal saw can cause irregular crack propagation and fractured bone chips, affecting the tissue removal process and postoperative recovery. Oscillating sagittal saws can also generate high levels of heat (e.g., up to 150° C. based on thermocouple and thermal imaging data), which can lead to tissue necrosis and delayed healing at the bone-implant interface.

Consequently, the surgical saw blade is usually externally water-cooled during the cutting process due to the impact of frictional heat on the biological potency of the bone. In addition, sclerotic bone lesions, which are regions of increased density within the bone, may further generate excessive heat during mechanical osteotomy leading to greater thermal necrosis and skiving of the blade leading to inaccurate cuts. The oscillating saw can also adversely affect the microstructure of bone tissue producing a significant amount of bone debris. The debris, or bone sequestra, can cause aseptic necrosis and retard bone regeneration by increasing the time for macrophages to cleanse the wound. The debris can also present a biohazard to the surgical staff (e.g., pulverized bone can form surgical burrs) if the debris becomes aerosolized, thereby increasing the risk of transmission of viruses such as SARS-CoV-2 in theatre.

Laser cutting techniques overcome many of these deficiencies due to their non-contact athermal nature. However, current laser systems, which are tailored for oral/maxillofacial surgery, adopt a Gaussian focused beam spot approach, Consequently, they resect bone relatively slowly as compared to conventional non-laser systems (e.g., less than 1 mm³/s). Moreover, laser systems typically have severe depth limitations (e.g., less than 21 mm) with respect to the amount of bone that can be resected. This limits their application to shallow osteotomies.

As such, it would be advantageous to have a handheld laser resection device, which builds upon the advantages of typical laser systems but utilizes optical-fiber laser technology to provide a rate of resection and a depth of resection that is comparable to mechanical resection devices.

### SUMMARY

In some embodiments, a robotically controlled laser resection device includes a handheld housing containing elements including a treatment laser configured to generate a treatment laser beam, a laser scanner configured to direct the treatment laser beam, a water nozzle configured to emit a laminar waterjet and an aperture configured to emit the treatment laser beam within the laminar waterjet; and; and a tracking marker interfaced to the handheld housing.

In some embodiments, the device further includes a handle and a gross positioning actuator; wherein the gross positioning actuator provides two degrees of freedom between the handle and the handheld housing.

In some embodiments, the device further includes a fine positioning actuator position to the laser scanner.

In some embodiments, the device further includes an inertial measurement unit, wherein the fine positioning actuator is configured to adjust the positioning of the treatment laser beam based on detected movement by the inertial measurement unit.

In some embodiments, the device further includes a laser emitter within the visible spectrum configured to aid in targeting the treatment laser beam.

In some embodiments, the device further includes a laser emitter configured to measure a distance between the device and a target surface.

In some embodiments, the device further includes an ultrasound transducer configured to detect acoustic waves generated by optical excitation of a target.

In some embodiments, the device further includes optical elements configured to shape the treatment laser beam into a top-hat beam.

In some embodiments, the treatment laser is an erbium yttrium-aluminum-garnet laser.

In some embodiments, the device further includes a tip, wherein the treatment laser produces a resection larger than the tip such that the tip can enter the resection.

In some embodiments, the tip is configured to be removeable and disposable.

In some embodiments, the device further includes an extraction system including a pressurized microcavity running parallel to the treatment laser beam.

In some embodiments, a surgical laser resection system includes a handheld device including a treatment laser configured to generate a treatment laser beam, a laser scanner configured to direct the treatment laser beam, a water nozzle configured to emit a laminar waterjet, an aperture configured to emit the treatment laser beam within the laminar waterjet, and a tracking marker; a control unit configured to control the treatment laser and control water flow to the water nozzle; a tracking system configured to detect a position of the tracking marker; a power generator electrically coupled to the treatment laser; and a cable harness interfacing the control unit and the power generator to the handheld device.

In some embodiments, the handheld device further includes a handle and a gross positioning actuator, wherein the gross positioning actuator provides two degrees of freedom of movement between the handle and the aperture.

In some embodiments, the system further includes an external control device configured to enable/disable the treatment laser.

In some embodiments, the control unit is configured to automatically disable the treatment laser based on achieving a predetermined resection depth.

In some embodiments, the handheld device further includes a fine positioning actuator configured to position the laser scanner.

In some embodiments, the handheld device further includes an inertial measurement unit, wherein the fine positioning actuator is configured to adjust the positioning of the treatment laser beam based on detected movement by the inertial measurement unit.

In some embodiments, the handheld device further includes a laser emitter within a visible spectrum configured to aid in targeting the treatment laser beam.

In some embodiments, the handheld device further includes a laser emitter configured to measure a distance between the device and a target surface.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical data server via a network in accordance with an embodiment.

FIG. 3 illustrates seven bone cuts performed in a typical total knee arthroplasty (TKA) procedure in accordance with an embodiment.

FIG. 4 illustrates the results of machining-induced thermal damage in cortical bone.

FIG. 5 illustrates a laser system that focuses the laser beam into a waterjet nozzle “Water-jet laser guidance” in accordance with an embodiment.

FIG. 6 illustrates the laser system's capabilities at producing deep line cuts in accordance with an embodiment.

FIG. 7 illustrates the laser system's capabilities at producing cylindrical holes, in accordance with an embodiment.

FIG. 8 illustrates non-contact/non-planar bulk bone shaping of the femoral condyles and tibial plateau in accordance with an embodiment.

FIG. 9 depicts a laser absorption chart.

FIG. 10A illustrates a comparison between an unshaped and shaped beam in accordance with an embodiment.

FIG. 10B illustrates a comparison between an unshaped and shaped beam with respect to ablation limit in accordance with an embodiment.

FIG. 11 depicts a robotic controlled laser-in-handheld control system in accordance with an embodiment.

FIG. 12 illustrates a schematic cross-sectional view of the fully integrated water-assisted handheld laser beam delivery system in accordance with an embodiment.

FIG. 13 illustrates a schematic cross-sectional view of the cable harness in accordance with an embodiment.

FIG. 14 illustrates a schematic cross-sectional view of the fully integrated robotic controlled laser-in-handheld control system in accordance with an embodiment.

FIG. 15 illustrates a block diagram of the robotically controlled handheld laser control system in accordance with an embodiment.

FIG. 16 illustrates a schematic cross-sectional view of the fully integrated handheld laser beam delivery system highlighting multi-level actuation in a handheld device in accordance with an embodiment

FIG. 17 illustrates a robotically controlled laser handpiece controlling the depth of a laser resection in accordance with an embodiment.

FIG. 18A illustrates a water-cooling system in accordance with the waterjet-laser guidance embodiment.

FIG. 18B depicts calculated transmission percentage through water in accordance with an embodiment.

FIG. 19 depicts a robotically controlled laser handheld with an external laser in accordance with an embodiment.

FIG. 20 depicts a ferrule connector for connecting to a multiplexed fiber end effector in accordance with an embodiment.

FIG. 21 depicts the robotically controlled laser end effector consisting of the piezoelectric transducer that supports vibration-assisted laser cutting in accordance with an embodiment.

FIGS. 22A-C illustrate example end 2201 of an end effector and the resultant homogenous intensity distribution in the bone 2203 created by the overlapping fiber lasers in accordance with an embodiment.

FIG. 23 illustrates the relative timing of a laser pulse and coolant delivery in accordance with an embodiment.

FIG. 24 depicts an Optoacoustic Computed Tomography (OACT) assembly in accordance with an embodiment.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

### Definitions

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of BLUE BELT TECHNOLOGIES, INC. of Pittsburgh, PA, which is a subsidiary of SMITH & NEPHEW, INC. of Memphis, TN.

### CASS Ecosystem Overview

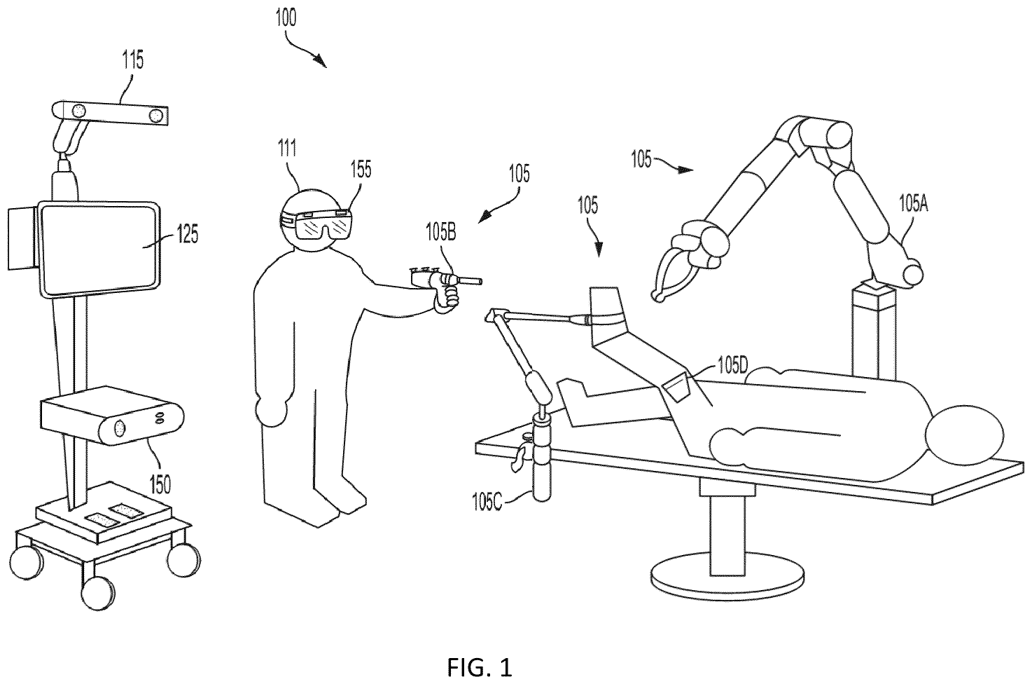

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA) or THA. For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a Robotic Arm 105A. While one Robotic Arm 105A is illustrated in FIG. 1, in some embodiments there may be multiple devices. As examples, there may be one Robotic Arm 105A on each side of an operating table T or two devices on one side of the table T. The Robotic Arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105C may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 105D can be formed integrally as part of the Effector Platform 105 or Robotic Arm 105A or cutting guides can be separate structures that can be matingly and/or removably attached to the Effector Platform 105 or Robotic Arm 105A. The Effector Platform 105 or Robotic Arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including, without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the Robotic Arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the Robotic Arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one- or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general-purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a Robotic Arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The Robotic Arm 105A may have multiple degrees of freedom (like a Spider device) and have the ability to be locked in place (e.g., by a press of a button, voice activation, a surgeon removing a hand from the robotic arm, or other method).

In some embodiments, movement of the Robotic Arm 105A may be effectuated by use of a control panel built into the robotic arm system. For example, a display screen may include one or more input sources, such as physical buttons or a user interface having one or more icons, that direct movement of the Robotic Arm 105A. The surgeon or other healthcare professional may engage with the one or more input sources to position the Robotic Arm 105A when performing a surgical procedure.

A tool or an end effector 105B attached or integrated into a Robotic Arm 105A may include, without limitation, a burring device, a scalpel, a cutting device, a retractor, a joint tensioning device, or the like. In embodiments in which an end effector 105B is used, the end effector may be positioned at the end of the Robotic Arm 105A such that any motor control operations are performed within the robotic arm system. In embodiments in which a tool is used, the tool may be secured at a distal end of the Robotic Arm 105A, but motor control operation may reside within the tool itself.

The Robotic Arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the Robotic Arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the Robotic Arm 105A can be moved by hand (e.g., by the surgeon) or with internal motors into its ideal position and orientation for the task being performed. In some embodiments, the Robotic Arm 105A may be enabled to operate in a “free” mode that allows the surgeon to position the arm into a desired position without being restricted. While in the free mode, the position and orientation of the Robotic Arm 105A may still be tracked as described above. In one embodiment, certain degrees of freedom can be selectively released upon input from user (e.g., surgeon) during specified portions of the surgical plan tracked by the Surgical Computer 150. Designs in which a Robotic Arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A Robotic Arm 105A or end effector 105B can include a trigger or other means to control the power of a saw or drill. Engagement of the trigger or other means by the surgeon can cause the Robotic Arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the Robotic Arm 105A or end effector 105B in an automatic mode that brings the robotic arm or end effector into the proper position with respect to the patient's anatomy in order to perform the necessary resections. The CASS 100 also can place the Robotic Arm 105A or end effector 105B in a collaborative mode that allows the surgeon to manually manipulate and position the robotic arm or end effector into a particular location. The collaborative mode can be configured to allow the surgeon to move the Robotic Arm 105A or end effector 105B medially or laterally, while restricting movement in other directions. As discussed, the Robotic Arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the Robotic Arm 105A or robotically controlled end effector 105B can be controlled remotely by a surgeon or other medical professional using a control mechanism separate from the robotic arm or robotically controlled end effector device, for example using a joystick or interactive monitor or display control device.

The examples below describe uses of the robotic device in the context of a hip surgery; however, it should be understood that the robotic arm may have other applications for surgical procedures involving knees, shoulders, etc. One example of use of a robotic arm in the context of forming an anterior cruciate ligament (ACL) graft tunnel is described in WIPO Publication No. WO 2020/047051, filed Aug. 28, 2019, entitled “Robotic Assisted Ligament Graft Placement and Tensioning,” the entirety of which is incorporated herein by reference.

A Robotic Arm 105A may be used for holding the retractor. For example, in one embodiment, the Robotic Arm 105A may be moved into the desired position by the surgeon. At that point, the Robotic Arm 105A may lock into place. In some embodiments, the Robotic Arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The Robotic Arm 105A may also be used to help stabilize the surgeon's hand while making a femoral neck cut. In this application, control of the Robotic Arm 105A may impose certain restrictions to prevent soft tissue damage from occurring. For example, in one embodiment, the Surgical Computer 150 tracks the position of the Robotic Arm 105A as it operates. If the tracked location approaches an area where tissue damage is predicted, a command may be sent to the Robotic Arm 105A causing it to stop. Alternatively, where the Robotic Arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the Robotic Arm 105A may be used to hold a cup impactor at a desired angle or orientation during cup impaction. When the final position has been achieved, the Robotic Arm 105A may prevent any further seating to prevent damage to the pelvis.

The surgeon may use the Robotic Arm 105A to position the broach handle at the desired position and allow the surgeon to impact the broach into the femoral canal at the desired orientation. In some embodiments, once the Surgical Computer 150 receives feedback that the broach is fully seated, the Robotic Arm 105A may restrict the handle to prevent further advancement of the broach.

The Robotic Arm 105A may also be used for resurfacing applications. For example, the Robotic Arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the Robotic Arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

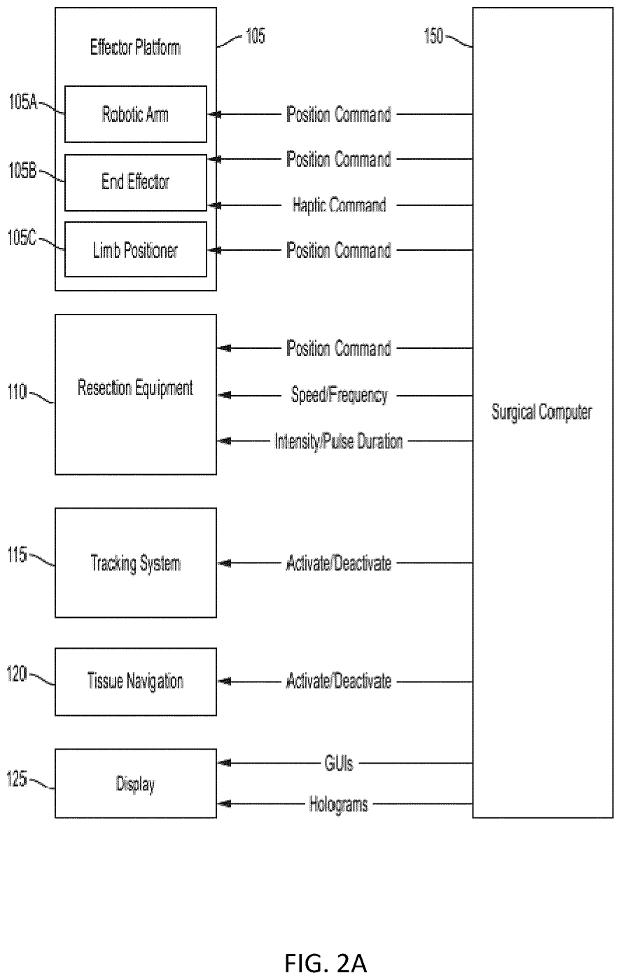

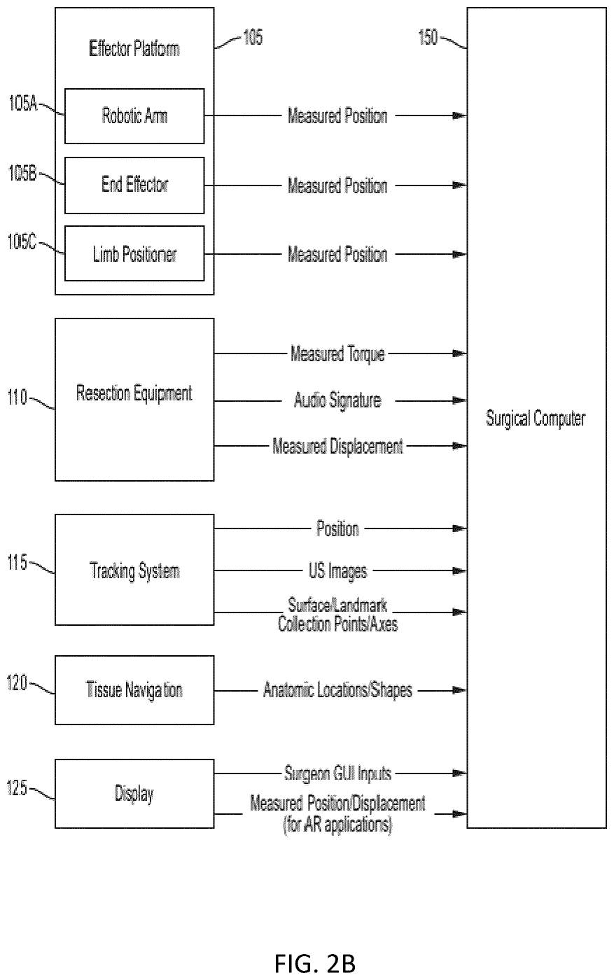

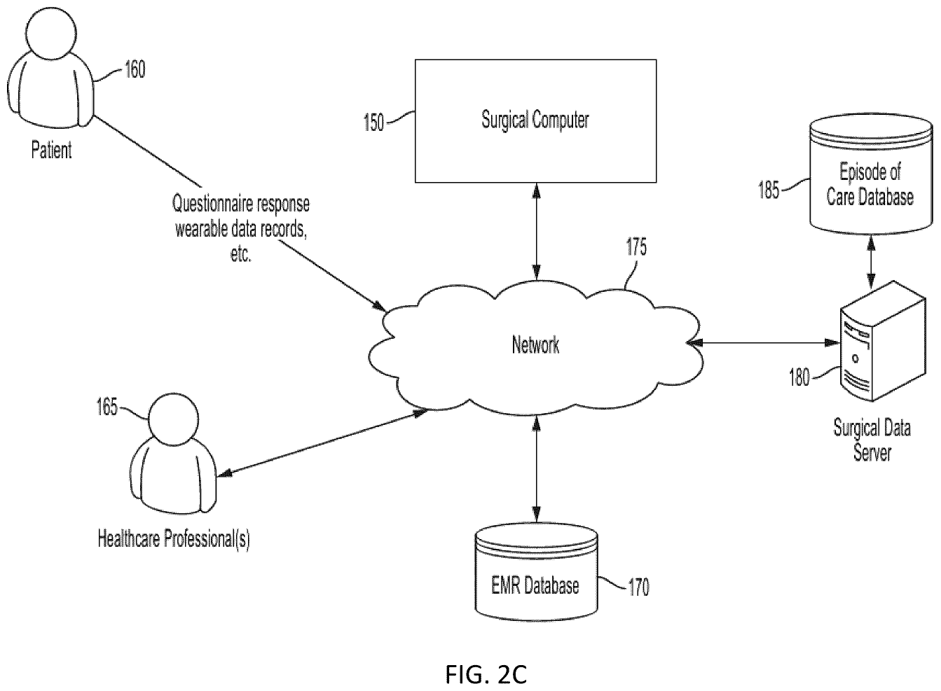

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 111 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner: the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon 111 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre- and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft HoloLens™ or Magic Leap One™, the Surgical Computer 150 may use the HoloLens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data.

In general, the Surgical Computer 150 may serve as the central point where CASS data is collected. The exact content of the data will vary depending on the source. For example, each component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively, or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. patent application Ser. No. 16/847,183, filed Apr. 13, 2020, published as U.S. Publication No. 2020/0243199, and entitled “METHODS AND SYSTEMS FOR PROVIDING AN EPISODE OF CARE,” the entirety of which is incorporated herein by reference.

### Orthopedic Oscillating Saws

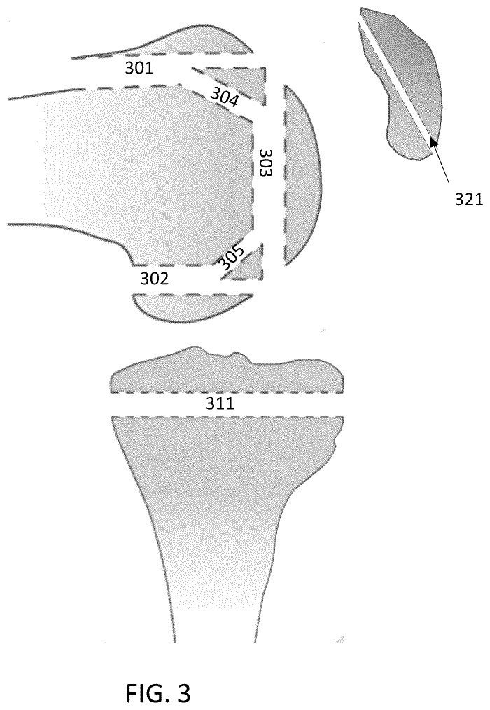

FIG. 3 illustrates the seven bone cuts in a typical total knee arthroplasty (TKA) procedure in accordance with an embodiment. The seven bone cuts can include the distal femur 303, anterior femur 301, posterior femur 302, anterior chamfer 304, posterior chamfer 305, tibia 311, and patella 321 cuts. Planar cuts can be as deep as 50 mm for the tibia 311, 30 mm for the five box cuts of the femur 301-305 and 20 mm for the patella 321 for a total knee replacement surgery. The anterior femoral cut 301 can set the femoral component rotation, which can have a direct effect upon patellar tracking and gap balancing. The posterior condyle 302 and tibial 311 cuts can affect the flexion gap.

Orthopedic oscillating saws are widely used for plane processing during joint replacement procedures with bone removal rates of approximately 5 mm³/s. However, a sagittal saw can cause irregular crack propagation and fractured bone chips, which in turn affects the tissue removal process and postoperative recovery.

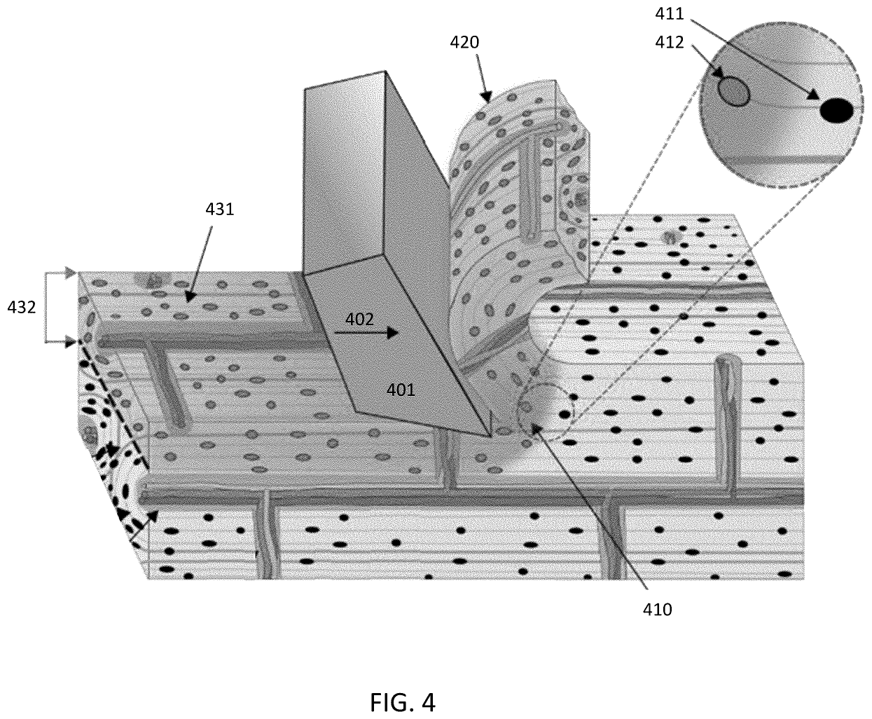

FIG. 4 illustrates the results of machining-induced thermal damage in cortical bone. Oscillating sagittal saws can generate high levels of heat (e.g., up to 150° C.), which can lead to tissue necrosis and delayed healing at the bone-implant interface. As the cutting edge 401 removes material 420, it leaves behind a machined surface 431 heated by the dissipation of the cutting energy 402. The heat is focused at the tip 410 of the cutting edge 401. The heat propagates deeper into the bone and, if the temperature is high enough (e.g., at least 47° C.), causes osteocyte necrosis 432. In FIG. 4, living cells 411 and dead cells 412 are differentiated.

Consequently, the saw blade can be externally cooled (e.g., through water) during the cutting process to lessen the effect of frictional heat on the biological potency of the bone. In addition, sclerotic bone lesions, which are regions of increased density within the bone, may further generate excessive heat during mechanical osteotomy, which may lead to greater thermal necrosis. The oscillating saw can also adversely affect the microstructure of bone tissue by producing a significant amount of bone debris. The bone debris, or sequestra, can cause aseptic necrosis, which can retard bone regeneration by increasing the time for macrophages to cleanse the wound. The bone debris can also present a biohazard to the surgical staff in a similar way to pulverized bone from surgical burrs. For example, if the debris becomes aerosolized, it can increase the risk of transmission of viruses such as SARS-COV-2 to the patient and theatre team.

### Laser Resection

Laser resection is an attractive alternative to mechanical resection of bone that offers the following benefits: high precision cuts, clean cuts, contact-free cuts eliminating mechanical loading and residual stresses, flexible cut geometries, an absence of vibration (e.g., with robotic control), an absence of cutting guides or templates, an absence of metal debris from the tool, a low roughness of cut surfaces, an absence of a smear layer on the osteotomy edges reducing healing time, and reduced damage to the surrounding soft tissues.

Despite these potential benefits, the widespread adoption of laser-assisted tissue resection has been typically limited to ophthalmology, oral and cranio-maxillofacial osteotomies due to the limitations of focused beam approaches. The main drawback of laser ablation, especially in comparison with mechanical cutting instruments, is a relatively low processing and cutting speed, and a lack of an ability to create planar cuts. The amount of bone removed by a single laser pulse is dependent upon the laser parameters and the composition of the bone and the ability to maintain efficient cutting at greater depths.

The absence of safe robotic guidance for controlling the position of the laser beam is also a limitation of most commercial systems. Robotic control can provide more efficiency and safety and the possibility of performing more complex surgical procedures (e.g., a TKA, Unicompartmental Knee Replacement (UKR), Total Hip Replacement (THR), and Total Shoulder Replacement (TSR)). However, an adequate depth feedback control system is typically absent with conventional laser resection systems. A properly implemented depth feedback control system may help protect vital tissues that could otherwise be contacted by the laser during the ablation process. A feedback control system could include differentiation feedback. For example, the system could provide a visual (e.g., on a display or via a light on or off the tool), haptic, or audio cue to the operator based on the differentiation of different tissues. Furthermore, the device could be configured to cease one or more or more operations (e.g., shut off the laser and/or waterjet) on detection of one or more specific tissues or navigate around said tissues.

The laser ablation system may also include a system that provides optimal delivery of concomitant “in-line” water irrigation to maintain a clean, safe working space, an effective ablation rate in the event of water or debris accumulation that could reduce the incidence energy of the laser, and a sufficient supply of water to prevent the carbonization of the tissue. Currently, there are two approaches; water-assisted laser cutting and waterjet-laser guidance. The effective ablation rate is referred to herein as the ablation threshold, which is the minimum fluence (i.e., energy per unit area) required for removal of the bone material. In some embodiments, with a laser fiber setup, the minimum fluence can be 7 J/cm² assuming a numerical aperture of 2×0.45 mm, 350 mJ per fiber and a distance of 2 mm from the bone surface.

The intensity of the laser beam becomes weaker as it propagates deeper inside a planar cut. However, the beam intensity can be improved by using waterjet guided laser (WJGL) machining or using in-line water-assisted cooling to remove the bone debris after each pulse.

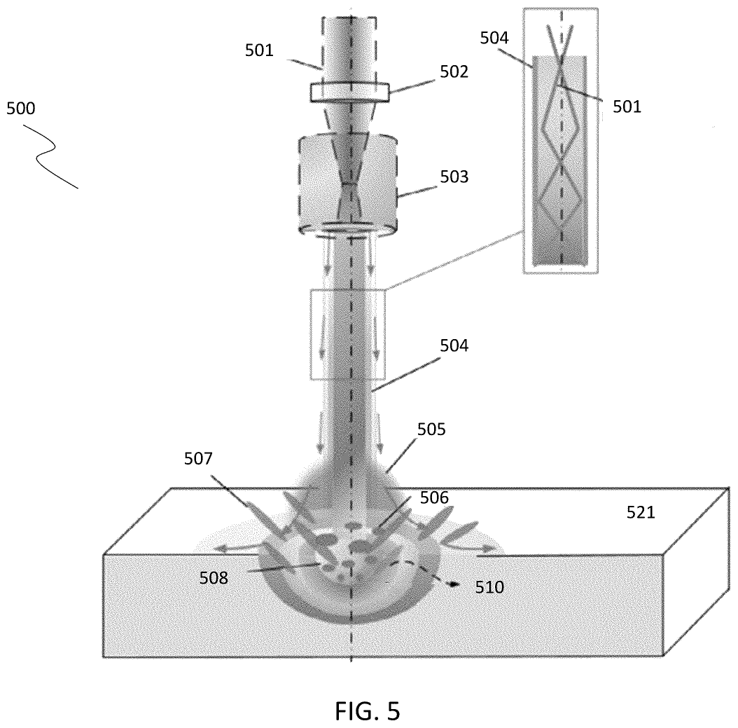

FIG. 5 illustrates a laser system 500 that focuses a laser beam 501 into a waterjet nozzle 503. The laser system 500 can use a waterjet beam 504 to guide the laser beam 501 similarly to a glass fiber to help achieve more accurate cutting under an increased working distance (e.g., 75 to 125 mm). The laser beam can be focused, using a lens 502, into a waterjet nozzle 503 and guided by a laminar waterjet 504 where it undergoes total internal reflection at the water/air boundary, leading to a long working distance without the beam 501 defocusing. In this scenario, while bone removal is dominated by the laser ablation through the formation of the melt pool 508, vapor 506, and plasma plume 505, the waterjet 504, with its high heat capacity, can significantly influence the thermodynamics and kinetics of the ablated matter 507, and provide good heat management and debris removal from the work zone. Hence, as compared to the laser ablation in air, where a great proportion of the heat can penetrate into a deep bulk area (e.g., >1 mm) with drastic thermal effects, the WJGL process provides a smooth cut and localized heat influence 510.

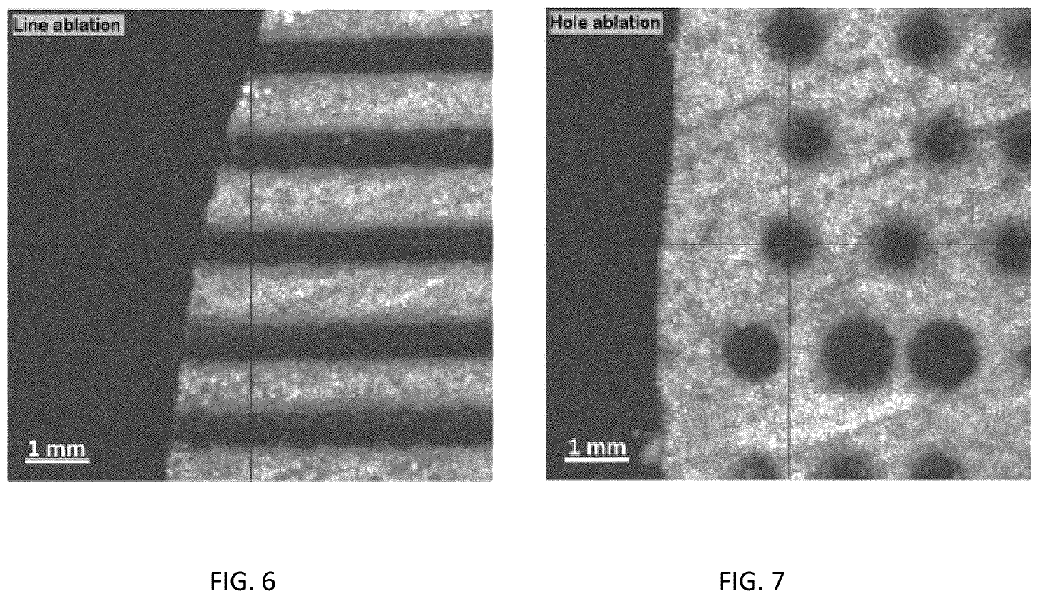

The laser system 500 can achieve both deep (e.g., 50 mm) line (i.e., planar) cuts, as depicted in FIG. 6, and cylindrical holes, as depicted in FIG. 7, depending on the settings of the laser 500. A combination of these techniques may facilitate the attachment of an implant onto the resected bone surfaces.

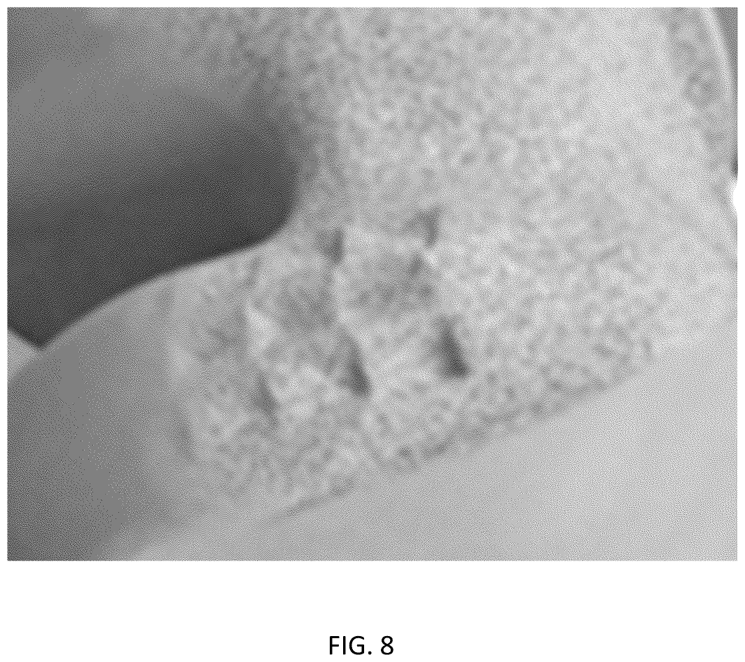

A laser system 500 may also be used for non-contact bulk bone shaping/resurfacing of the femoral condyles and tibial plateau within a sub-millimeter level of accuracy, as illustrated in FIG. 8. Specific features on the bone surface (e.g., the implant bed) amenable to a laser in a secondary cut could include a variety of cuts (e.g., undercuts, dovetails, spline geometries, etc.). The improved surface processing may allow for a better fit between the bone and the implant and could improve implant stabilisation during the initial fixation period. Surface morphing of the bone could accommodate lower profile implant geometries. As such, precise bone shaping may reduce the need for non-biological fixation methods (e.g., bone cement). Using the systems described herein fixation may be entirely cementless.

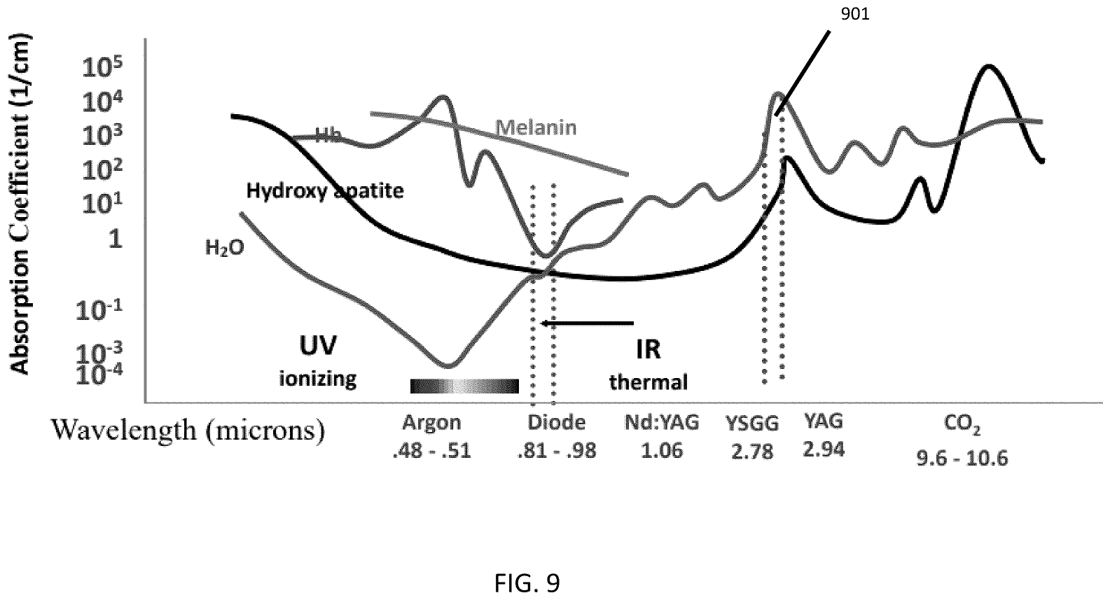

Several types of lasers in the mid infrared (IR) range are commercially available for bone resection including CO2 and erbium-doped yttrium-aluminum-garnet (Er: YAG) lasers. FIG. 9 depicts a laser absorption chart. According to some metrics, Er: YAG produces lasers of the least invasive wavelength. With Er: YAG laser sources, laser pulse durations between 100 and 500 us were below the thermal relaxation time so that hard tissue ablation was possible. Furthermore, both water and hydroxyapatite, which are the two main chromophores in bone, have high absorption peaks 901 near 3 μm, As such, the mechanism of bone ablation is thermally mediated by explosive pyrolysis of the substrate. Control of the extent of lateral damage during laser irradiation can be based primarily on the speed of the laser application (i.e., a faster pulse results in less time for conduction into adjacent tissue).

Er: YAG laser bone ablation, as compared to burr drilling, can create an irregular micro-surface without a smear layer, increasing fibrin attachment and aggregation of red blood cells due to the mechanical trapping effect of the laser roughened bone surface. The resulting surface is advantageous for blood clot attachment, thereby helping to promote new bone formation.

Several laser pulse regimes are available (e.g., nanosecond, picosecond, and femtosecond) for ablating hard tissues. Lasers with pulse durations in the femtosecond to picosecond range can be restricted to ablating bone tissues in the millimeter range without waterjet assistance. Microsecond lasers, e.g. 50-500 us can be tailored to produce deeper planar cuts in bone. In particular, because the pulse duration is longer than the thermal relaxation time of the tissue, the ablation area remains relatively cold due to the ablation mechanism.

### Laser Beam Shaping

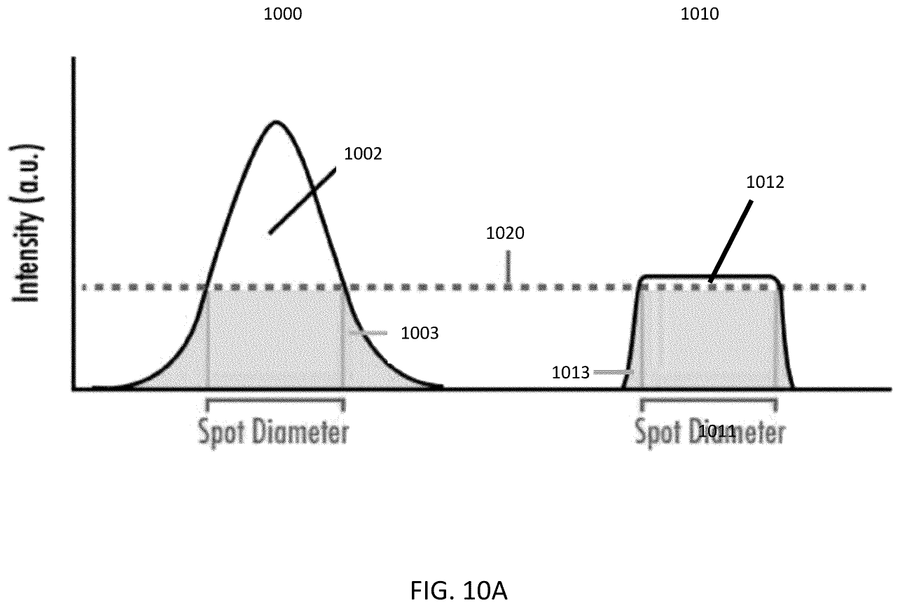

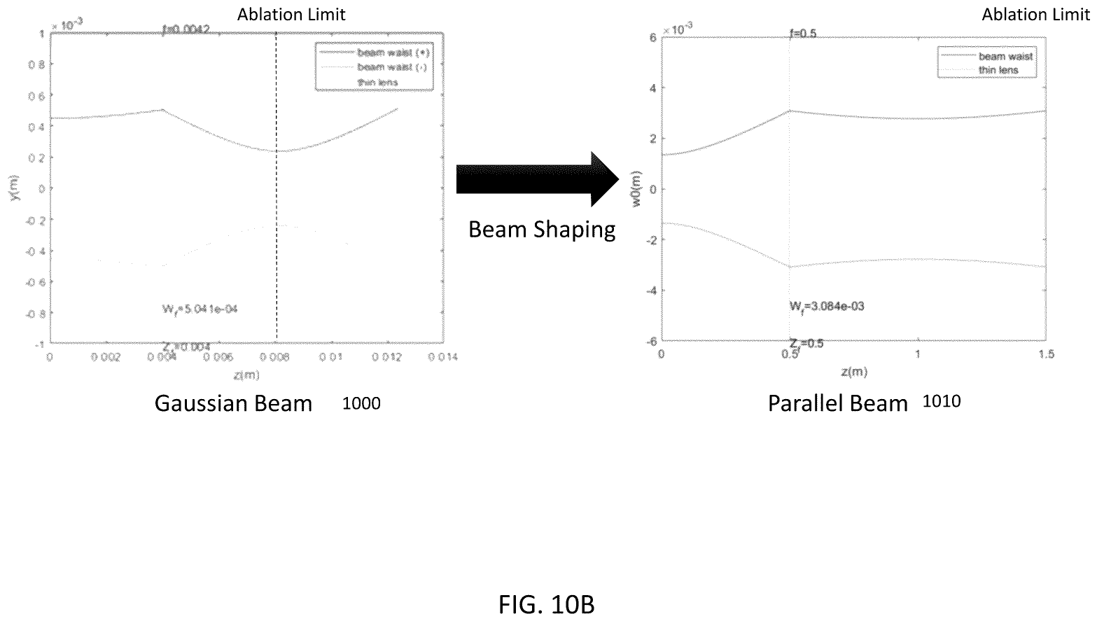

The output intensity profile of a laser beam can have a Gaussian profile. The corresponding ablation profile follows the reciprocal of the intensity profile, providing a conical shape. This intensity profiles leads to the laser delivering less energy to the deeper points within the cut as the walls of the cut spatially filter the beam, resulting in a limited cutting depth. Additionally, there is limited diffractive element production in the mid-IR region. As a result, the straight-walled cutting approach has typically not been applied to biological tissues. FIGS. 10A and 10B illustrate a comparison between an unshaped 1000 and shaped 1010 beam in accordance with an embodiment. By obtaining low-intensity side lobes, the coupling efficiency can be increased, and the highest possible deliverable average energy can be provided. Beam shaping also results in straight-walled cuts. Once the parallel beam is obtained, the laser beam may then be directed into the diffractive element. The diffractive element is designed to shape the intensity distribution of the laser (e.g., Er: YAG laser).

Multiplexing an array of overlapping fibers (e.g., engineered sapphire fibers) embedded into an end effector can steer and shape a line-shaped laser beam increasing the speed of ablation. By keeping a constant distance between the laser aperture and the surface, the depth of focus for ablation depth may not be limited. The output of the fibers can be arranged to obtain overlapping regions, which can increase the rate of ablation.

In some embodiments, high-energy Er: YAG lasers may have a wavelength of 2940 nm. Typical flashlamp-pumped systems can produce highly divergent, low-quality, unshaped output beams 1000. Typically, an unshaped output beam 1000 has an approximately Gaussian intensity distribution. The high intensity at the center of the unshaped output beam 1000 is well above the ablation threshold 1020. As such, the unshaped output beam 1000 expends superfluous energy 1002. The superfluous energy 1002 may also cause damage to any medium (e.g., a fiber) used to deliver the beam 1000. Furthermore, additional heat energy 1003 is delivered to the resection site, increasing damage to the bone.

The amount of superfluous energy 1002 and heat energy 1003 can be minimized using beam shaping. A shaped beam 1010 can be shaped using a set of optics or diffractive elements to produce a “top-hat beam shape” or line beam. The resulting shaped beam 1010 can have greatly reduced superfluous energy 1012 and heat energy 1013 as compared to the unshaped beam 1000. A shaped beam 1010 limited to a specific spot diameter can achieve straighter walled cuts.

### Robotically Controlled Laser-in-Handpiece

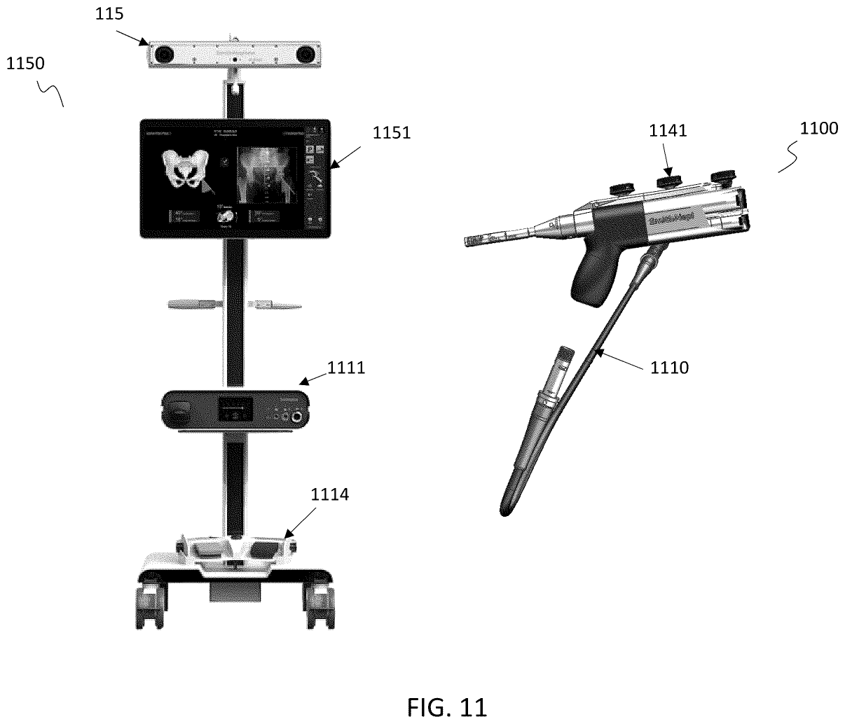

A robotically controlled laser-in-handpiece is configured to enable a real-time navigated execution of predefined geometries tailored for surgical procedures including joint replacement surgery by virtue of the handheld tool. The handheld tool can be programmed to offer selective ablating or cutting of a targeted area of bone with an embedded laser. FIG. 11 depicts a robotic controlled laser-in-handheld control system in accordance with an embodiment. The handpiece 1100 may interface to a base unit 1150 using the cable harness 1110. The cable harness 1110 may be terminated by any known coupling mechanism. In some embodiments, the base unit 1150 includes one or more of a power source, a fluid reservoir, a cooling fluid source, a vacuum system, an external tracking system 115, a controller 1114, a display 1151, and a control unit 1111. One or more sub-components located in the base unit 1150 may be electrically connected to the handpiece 1100. The handpiece 1100 can include one or more tracking markers 1141 configured to allow the handpiece 1100 to be trackable by the tracking system 115.

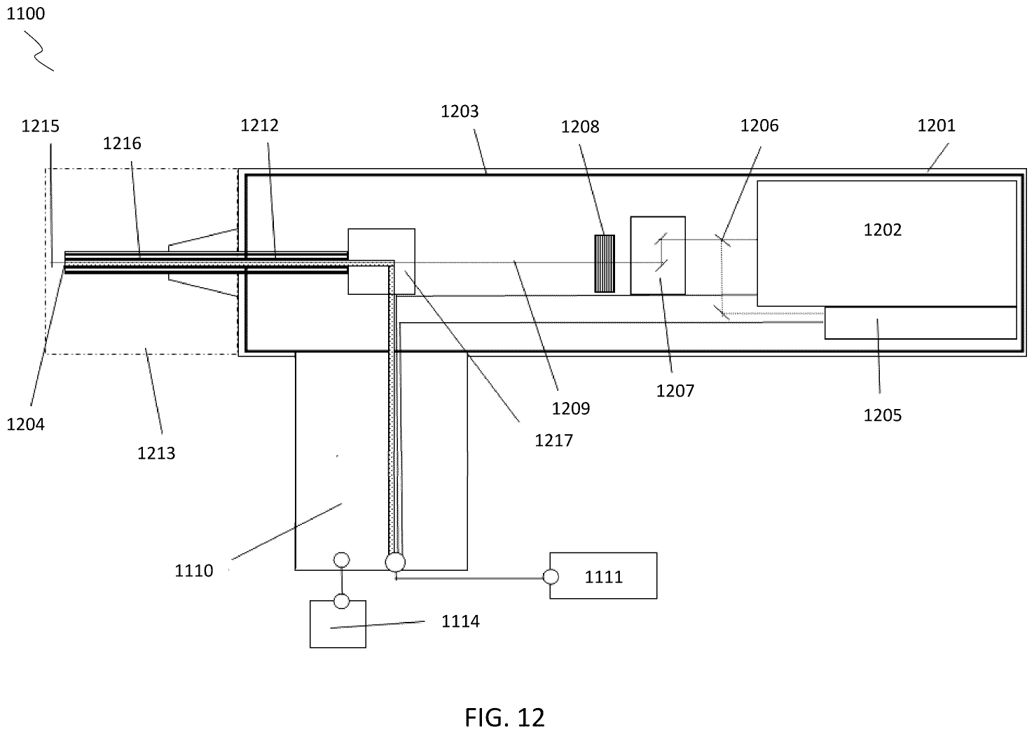

FIG. 12 illustrates a schematic cross-sectional view of a handheld laser beam delivery system and control unit 1111 in accordance with an embodiment. The system 1100 can produce a succession of pulses of generated radiation with an energy level, pulse duration, and repetition rate specified to cut or ablate bone material without causing harmful side effects and significant bone loss. The system 1100 can be configured to eliminate the need for a complex conveying system such as a long optic fiber or an articulated jointed arm.

In some embodiments, the handpiece 1100 houses the laser delivery optics, as indicated in FIG. 12. The handpiece 1100 may use a water-guided amplifying medium (i.e., a laminar waterjet) 1212 to create planar cuts and/or cylindrical holes in bone during joint arthroplasty with a focused IR laser light source (e.g., Nd: YAG). The laminar waterjet 1212 may provide collaborative effects in terms of cooling the site of the tissue ablation and guiding the laser beam to achieve more accurate cutting at an increased working distance (e.g., up to 50 mm). In some embodiments, the entire power generator is incorporated within the handpiece 1100. The treatment laser beam 1209 may be focused through the tip 1215 of the handpiece 1100 using a series of reflective mirrors 1207 disposed within the handpiece and a lens 1208 (e.g., an in-line miniaturized calcium fluoride (CaF2) lens) to deliver higher peak power to the tissue surface. In some embodiments, the laser beam 1209 is focused into a waterjet nozzle 1217. In some embodiments, the treatment laser beam 1209 is guided by a laminar waterjet 1212 where the treatment laser beam 1209 undergoes total internal reflection at the water/air boundary, enabling a long working distance from the tip aperture 1204 without the treatment laser beam 1209 suffering any significant defocusing. In this scenario, bone removal can be dominated by laser ablation through the formation of a melt pool, vapor and plasma plume. The tip 1215 can be configured to chase the cut into the patient bone, thus reducing depth limitations. In other words, the treatment laser 1209 may produce a resection larger than the tip 1215 such that the tip 1215 can enter the resection.

Although the laminar waterjet 1212, with its high heat capacity, can positively influence the thermodynamics and kinetics of the ablated matter by providing good heat management and cleaning the debris from the work zone, there is a risk of excess water build up in the cutting zone. In some instances, excess water can negatively impact ablation efficiency. To counteract this effect, the handpiece 1100 can be equipped with a series of pressurized micro-channels 1216 running parallel with the treatment laser beam 1209 to evacuate excess water at the cutting interface. The evacuation/suction system 1216 ensures that the treatment laser beam 1209 can reach the bone surface without encountering additional absorbing material by overcoming the shielding effect provoked by excess water and debris on the beam path. A control unit 1111 may control delivery of the flow of air, via one or more microchannels parallel to the treatment laser 1209, such that the flow of air is provided to the site of the ablated tissue at an appropriate rate during application of the laser pulse to the site.

Alternatively, liquid coolant is delivered to the site of the tissue ablation during a time period that does not overlap in time with delivery of the laser pulses to the site of the tissue ablation. The control unit 1111 may control delivery of the liquid coolant by means of a solenoid-operated valve.

In some embodiments, a handpiece housing 1201 houses a laser mechanism 1202 that includes a primary laser energy emitter, a power generator (e.g., 24V), and an electromagnetic shield within a small chamber in the handpiece. Integration of the laser emitter in the handpiece housing 1201 can simplify product manufacture, eliminate the need for frequent laser pass folding mirror adjustments, and minimize the risk of optical fiber cracking from operator movement. Alternatively, one or more components of the laser mechanism 1202 may be external (e.g., an external power source). When excited, the laser medium can radiate laser energy (i.e., a laser beam) to the treated tissue via an aperture 1204, from the laser tip 1215, in the handpiece 1100. This may minimize energy loss and enhance the laser's cutting capabilities.

The laser energy emitter may include a solid-state laser medium and a pump source. In some embodiments, the laser medium is a compact (e.g., 125 mm×70×55 mm) Class IV pulsed diode pumped solid state laser encased within the handpiece housing 1201. In some embodiments, the laser can be a Er: YAG laser (e.g., 2,940 nm wavelength), which is optimally absorbed in water and hydroxyapatite (i.e., the two main chromophores in bone). In various embodiments, the pump source can be a Xenon flash lamp, a Krypton flash lamp, or an array of semi-conductor laser diodes. The handpiece housing 1201 can include a secondary laser source 1205 for generating a visible aiming beam to align the handpiece with respect to the bone. For example, a low-power continuous-wave Class I green visualization laser can allow the surgeon to see the axis of the ablation beam both during and before activating the ablation. The handpiece 1100 may include a beam-combing window 1206 which allows light from the laser mechanism 1202 to pass through while reflecting the light from the secondary laser source 1205, which brings the laser beam from the laser mechanism 1202 and the laser beam from the secondary laser source 1205 into coaxial relation.

The handpiece housing 1201 may further include an RFID microchip that supports recording and calibration of the laser emitter. The RFID microchip may store calibration values for the laser mechanism 1202 and other components in the handpiece 1100.

The handpiece housing 1201 may further include a high-speed XY motorized laser scanner 1207. The high-speed XY motorized laser scanner 1207 can include two scanner mirrors, which can be commanded to tilt the laser beam along either the X axis, the Y axis, or both. As a result, the focused spot of the treatment laser will be shifted in either axis of the working plane, which is defined by a distance equal to the focal distance of the lens 1208. The treatment laser beam 1209 can sweep systematically along a pre-operatively defined osteotomy path at a defined speed while maintaining a consistent overlap between consecutive laser pulses. This continuous motion contributes to controlled limitations of the amount of energy delivered at a given location.

Locating the solid-state laser medium and a laser pump source in the handpiece housing 1201 increases the risk of producing undesired electromagnetic emission. Therefore, the handpiece 1100 can include an electromagnetic radiation shield 1203, which comprises at least one electrical conducting layer (e.g., copper and/or nickel) and at least two electrical insulating layers (e.g., a pliable polyimide film). The electromagnetic radiation shield 1203 partially covers the internal electrical components of the handpiece 1100 such as the laser emitter 1202 and related control circuitry. The electromagnetic radiation shield 1203 can reduce the average radiated electromagnetic emissions and increase the safety of the device for the surgeon, surgical staff, and the patient. For example, the electromagnetic radiation shield 1203 can reduce electromagnetic emissions to 5-10 dB. The electrical conducting layer can be a metal layer sandwiched between two isolating material layers. In some embodiments, the electromagnetic radiation shield 1203 is grounded. In some embodiments, the electromagnetic radiation shield 1203 can span from the handpiece 1100 to a control unit 1111 (i.e., shielding the cable harness 1110 electrically connecting the handpiece 1100 and the control unit 1111).

In some embodiments, the handpiece 1100 includes a laminar waterjet 1212 to cool the laser energy emitter. In some embodiments, the handpiece 1100 includes a disposable end piece section 1213 that is configured to be disposed of at the end of the usage, thereby reducing the cost of sterilizing the optical and mechanical assembly. In some embodiments, the handpiece 1100 is interfaced to an external controller 1114, such as a foot pedal, to generate a trigger signal to start the laser irradiation process. The controller 1114 may also initiate operation of the high-speed XY motorized laser scanner 1207 and/or water emission. The controller 1114 may be interfaced directly to the handpiece 1100 or indirectly through the control unit 1111.

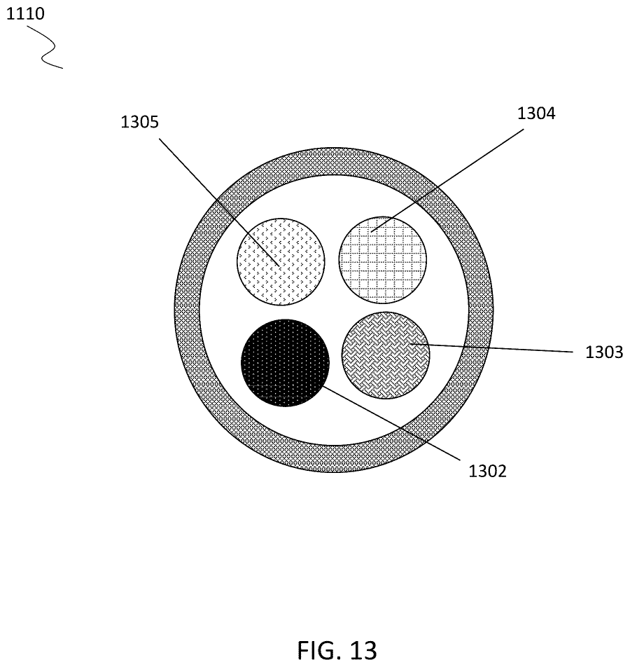

Referring to FIG. 13, a cable harness 1110 is depicted in accordance with an embodiment. The cable harness 1110 may connect the control unit 1111 to the handpiece 1100. In some embodiments, the cable harness 1110 includes a multi-luminal tube enclosing several lumina made of an extruded polymer. In some embodiments, the cable harness 1110 includes a single lumen tube having a lumen accommodating one or more separate tubes or conduits. In some embodiments, the cable harness enclosure is made of a semi-rigid or flexible material, such as a polymer (e.g., PVC). In some embodiments, the cable harness 1110 may accommodate additional cooling fluid conduits for cooling the laser emitter 1304, pressurized air for evacuating excess water at the cutting interface 1305, communication with the cooling fluid/air source 1303, and electrical power 1302.

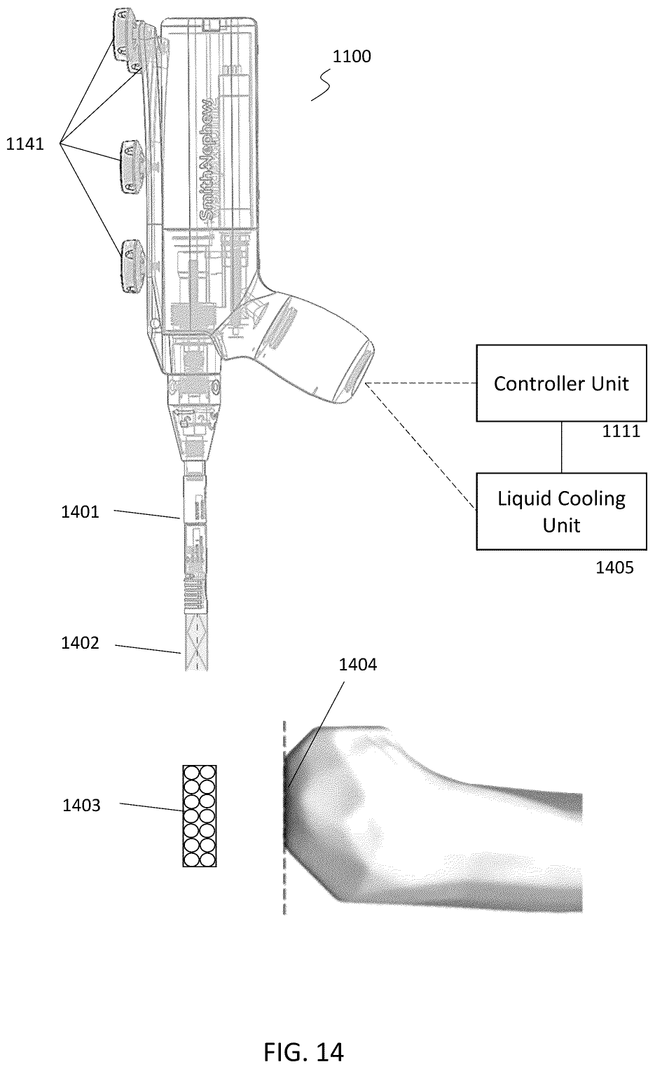

FIG. 14 depicts a cross-sectional view of the robotically controlled handheld laser system in accordance with an embodiment. In some embodiments, the handpiece 1100 interfaces with one or more components including the control unit 1111 and a liquid cooling unit 1405. The liquid cooling unit 1405 may provide fluid at a desired pressure and/or temperature to the handpiece 1100. The liquid cooling unit 1405 may be in operable communication with or integrated into the control unit 1111. A ruggedized housing 1401 may guide the laser beam inside a waterjet 1402 out of an aperture on the handpiece 1100. In some embodiments, the ruggedized housing 1401 may have a diameter of 10 mm. In some embodiments, the ruggedized housing 1401 may have a diameter between 15 and 25 mm. In some embodiments, a programmed outline 1403 may be filled with laser pulses to resect the bone 1404.

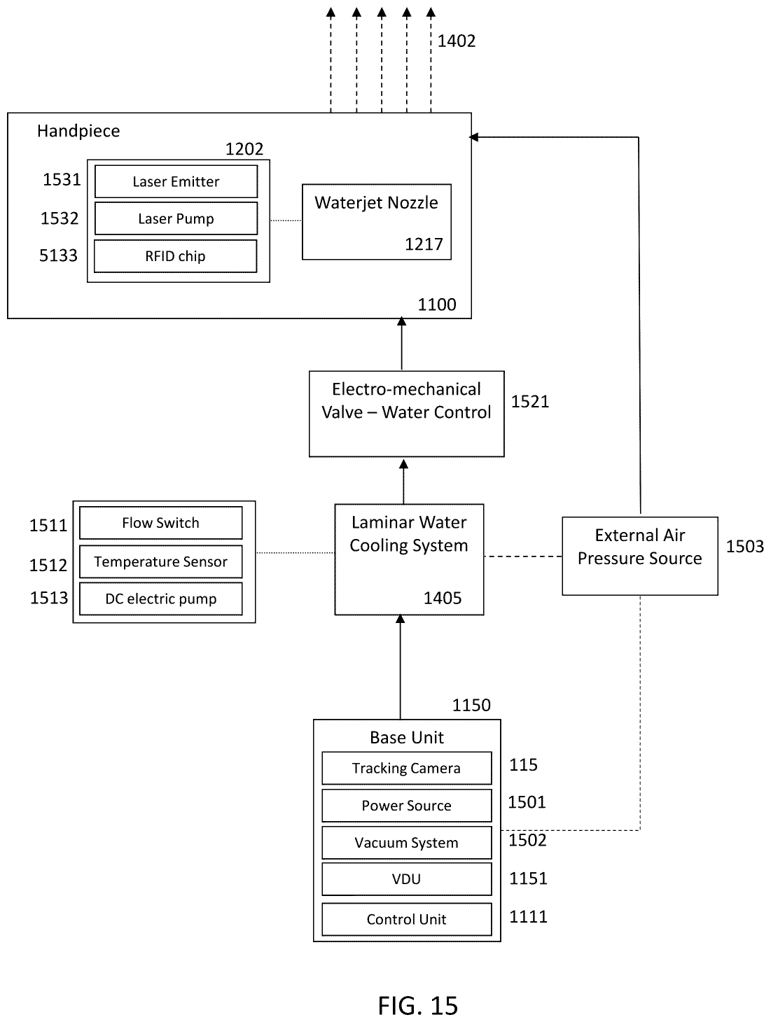

FIG. 15 illustrates a block diagram of the robotically controlled handheld laser control system in accordance with an embodiment. The handheld laser control system may include one or more external components, such as a tracking system 115, a power source 1501, a vacuum system 1302, a display 1151, a control unit 1111, an external air pressure source 1503, a liquid cooling system 1405, and/or a liquid flow control system 1521. One or more of the components may be interfaced to a base unit 1150 to simplify movement and installation of the handheld laser control system. The liquid cooling system 1405 may include one or more of a flow switch 1511, a temperature sensor 1512, a pump 1513, a reservoir, and a temperature control. Liquid/gas supply, control signals (e.g., digital or analog), and power can be delivered to the handpiece 1100 via one or more umbilical connections to the handpiece 1100.

The handpiece 1100 may include a laser 1202 and a waterjet nozzle 1217. The laser 1202 may include a laser emitter 1531, a laser pump 1532, and a control circuit/chip 1533. The handpiece 1100 may emit a laser beam within the waterjet 1402.

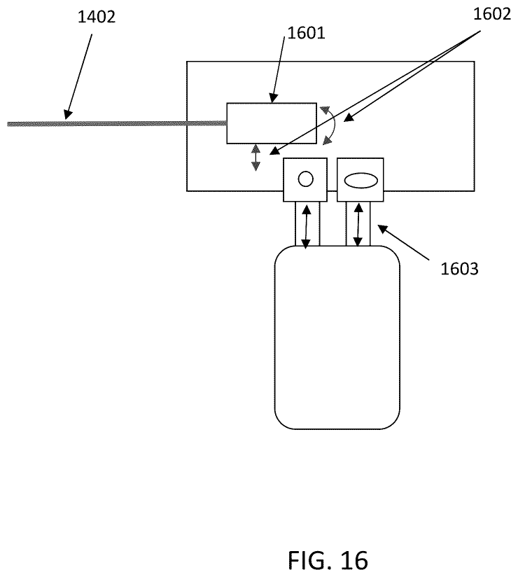

FIG. 16 illustrates multi-level actuation in a handheld device in accordance with an embodiment. The mechanisms for providing 2 DoF may be included in any of the handheld systems described herein (e.g., the handpiece presented in FIG. 12). The 2 DoF can be broken down into a translational component and the rotational component. Gross translation can be performed in the handle of the handpiece with one or two linear actuators 1603. Because the laser/waterjet 1402 collimator 1601 is a relatively small component, steering of the beam 1402 can be accomplished with the use of small micromotion actuators 1602. For example, the actuators 1602 can be based on piezo stacks, piezo “walkers,” or voice coil mechanisms. One advantage of separating the 2 DoF into translational and rotational components is the ability to break the motion down into gross and fine movements. Providing a gross position, via gross positioning actuators 1603, may place the mechanism close to the desired plane. The micro actuators 1602 may be used to accomplish fine positioning. For example, the micro actuators 1602 may be able to overcome movement that results from hand tremors of the operator. A handheld tool featuring gross actuators is described in WIPO Publication No. WO 2023/003849, filed Jul. 19, 2022, entitled “Surgical resection device and surgical computing device,” the entirety of which is incorporated herein by reference.

In addition to the use of a tracking system 115, an inertial measurement unit (IMU) may be included in the handpiece 1100 to achieve better dynamic performance. As described above, the handpiece 1100 may compensate for high frequency motion caused by hand tremors (e.g., by using the fine positioning actuators 1602). However, the camera frame rate may be relatively low and may be insufficient to track high frequency motion. Therefore, an IMU can be used to measure high frequency motion and provide feedback to the fine positioning actuators. Filtering techniques, such as an extended Kalman filter, can be used to fuse the gross positioning system and the fine positioning system.

As described herein, the handheld connected device includes a laser source, which emits the treatment laser beam. The laser pump source may include one or more flash lamps. In some embodiments, the flash lamps may include a flash lamp having a spectrum suitable to excite the laser medium (e.g., a Xenon or a Krypton flash lamp). In some embodiments, the laser pump source may be a semiconductor diode array providing sufficient power (e.g., 50 to 1000 W) to excite the laser medium. In some embodiments, pulsed infrared (IR) laser beams may be used to remove hard tissues instead of a mechanical drill or oscillating blade. In some embodiments, a fixed amount of material is removed per laser pulse. In some embodiments, an Er: YAG laser is tailored to the absorption peaks of water and hydroxyapatite, near 3 μm, which are the two main chromophores in bone. A continuous wave Class I green visualization laser can allow the surgeon to see the axis of the ablation beam before, during and after activating the ablation.

Laser parameters optimized for cutting bone may include some combination of the following: an average and high peak power of 90 and 500 W, respectively, whereby peak power can be inversely proportional to the repetition rate; a pulse duration between 100 us and 500 μs (i.e., the micro-second range), which is below the thermal relaxation time conducive for bone ablation; a high repetition rate having a frequency between 1 and 100 Hz, which is appropriate for allowing systematic robotic motion between consecutive pulses; a peak radiation intensity greater than 1011 W/cm²; a laser beam radius of 1.96 μm covering a 1 mm by 50 mm scan area; a working distance of 2 to 50 mm; a cutting width of 1 to 20 mm; a feed rate of 50 to 150 mm/s; and a laser fluence (i.e., intensity or ablation threshold) of 5 to 10 J/cm². The benefit of ultrafast laser machining can lie in the non-thermal nature of the ultrafast process (i.e., pulse durations are smaller than the thermal diffusion time).

In some embodiments, the handpiece 1100 can contain a red diode laser with a wavelength of 650 nm. The red diode laser may be used for aiming and aligning the targeted area with the area on the bone that needs to be ablated or cut. The apparatus can manoeuvre the focused red diode laser at a sufficient rate (e.g., 50 times per second) to form a visible standing image of the selected area boundaries that can be used by the operator to aim and align the area with the ablation site.

The system may also include an electric activating switch (e.g., a foot pedal) that the surgeon can use to activate the laser to ablate or cut the area. By activating the electric switch, the focused laser beam is directed to a first spot inside the selected area, and the laser direction is maintained at the spot for a predetermined time. In some embodiments, the laser may be focused on the first spot for 2.5 ms. Within this time, the laser may release one pulse. The user can repeat the ablation by using the same setting and activating the foot pedal again for another layer or changing the setting for a different shape or different size. Separate controls can be included for other systems, including liquid flow, air flow, and guidance laser activation. Alternatively, the control of one or more of these systems may be tied directly to the control of the treatment laser.

The handpiece 1100 may include an imaging optic system, which is designed to image and deliver the treatment laser emission. A CaF2 objective lens may be located in front of the laser source. A CaF2 lens exhibits negligible multiphoton absorption, which can enable higher peak power pulse delivery while focusing over a larger ablation field of view than, for example, a Zinc Sulphide (ZnS) lens. An optional near infrared (NIR) anti-reflection coating (e.g., a refractory oxide coating) may be used to counteract the Fresnel reflections off high refractive index lenses.

To reduce the incidence of thermal necrosis in the heat-affected zone, current laser systems may use water spray applied simultaneously with laser radiation in a similar way to mechanical instruments. The water spray may be useful for removing debris remaining from previous pulses but may block the laser radiation and decrease the ablation efficiency. Moreover, the intensity of the laser beam may become weaker as it propagates deeper inside a planar cut.

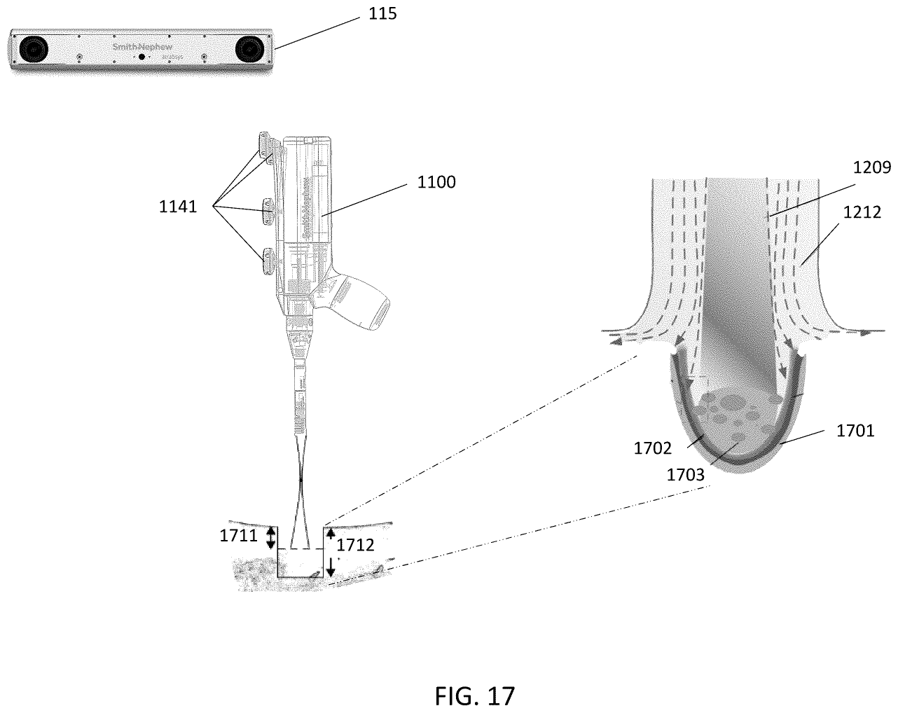

FIG. 17 illustrates a robotically controlled laser handpiece 1100 controlling the depth of a laser resection in accordance with an embodiment. The treatment laser beam 1209 can be transmitted through a laminar waterjet 1212 and can irradiate the material leading to some combination of melting 1701, vaporisation 1703, and ionization 1702 of the bone. The handpiece 1100 can be robotically controlled based on information received from an optical tracking system 115 to assist with the highly precise and functional bone cuts. The tracking system 115 may be used to determine the real-time position of the handpiece 1100 with respect to the targeted bone using one or more tracking markers 1141 (e.g., optical reflective markers) attached to the handheld pointer probe, patient anatomy, and the handpiece 1100. The CASS 100 monitors the position of the laser end effector with respect to the targeted bone. The system can use an external IR camera fixed at a known distance and orientation to provide real-time positions of the markers fixed to the targeted bone, to the laser handpiece 1100, or to a handheld pointer used during patient registration, planning and bone resection. The accuracy of the CASS 100 for identifying the position of a single marker depends on the position within the working volume. Based on one or more of the location of the handpiece 1100, the location of the bone, the operating characteristics of the laser, sensed cutting temperatures, a secondary laser configured for distance measurement, and optoacoustic/photoacoustic imaging, the CASS 100 can determine a current resection depth 1711. The handpiece 1100 may be configured to automatically perform an action when the current resection depth 1711 reaches a preprogramed planned resection depth 1712. For example, the handpiece 1100 may automatically deactivate the laser or retarget the laser as described herein. The preprogramed cut area may be of any shape (e.g., circle, square, rectangle, line, or triangle). In some embodiments, the shape of the cut area can be programmed to a size between 1 mm and 50 mm.

Manually positioning the laser/water beam accurately for a resection may be challenging. The handpiece 1100 may include one or more mechanisms for position and orientation control. With a laser, the cutting mechanism may be axisymmetric. As a result, providing 2 degrees of freedom (DoF) may be sufficient to keep the cutting axis in a desired plane.

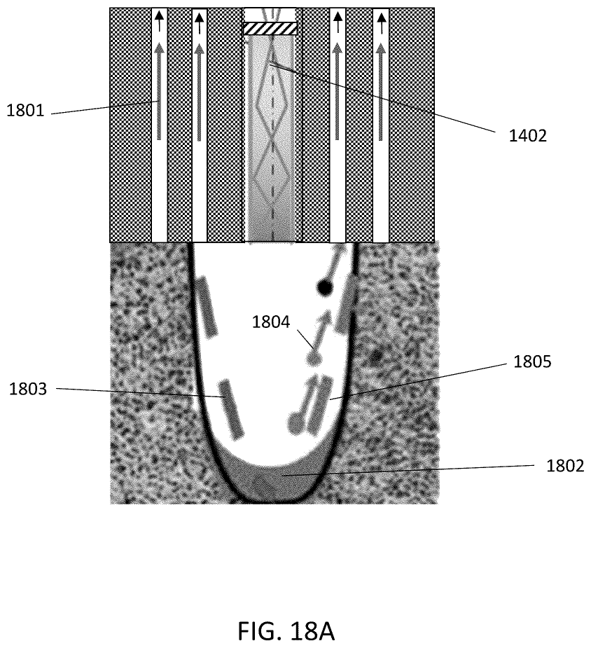

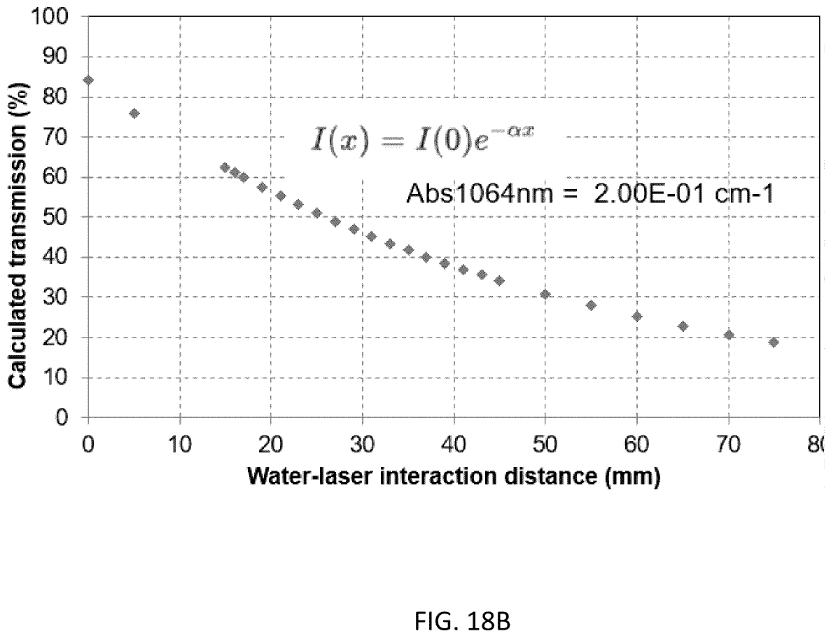

FIG. 18A illustrates a water-cooling system in accordance with an embodiment. The system can address beam propagation using a waterjet beam pumped at a sufficient pressure (e.g., approximately 0.5 MPa) to guide the laser 1402 similarly to a glass fibre to help achieve more accurate cutting under an increased working distance (e.g., 75 to 125 mm). The laser beam (e.g., a Nd: YAG laser with a wavelength of 1064 nm) can be focused into a waterjet nozzle and guided by a laminar waterjet. The laser beam may undergo total internal reflection at the water/air boundary, thereby enabling a long working distance without the beam defocusing.

One downside of using a continuous focused water spray can be that water strongly absorbs energy from an IR laser, as illustrated in FIG. 18B. As more water is used for cooling, more light is absorbed by the water, which increases with distance from the nozzle, thereby leading to decreased ablation efficiency. For example, approximately 80% of the IR laser is transmitted through 14 mm of water with an Nd: YAG laser. Water can also accumulate and block the energy of the laser source causing the tissue to carbonize. These issues can be significant in the case of low energy per pulse regimes and in the case of deep planar cuts (e.g., in the distal femur), which may be filled by water. The water spray in the air gap between the laser output and the tissue may also partially block the laser beam. The handpiece 1100 may be extended with a tip to be less sensitive to water spray occlusion because the air gap is shorter, but the spray may accumulate and create a large water drop on the tip. Therefore, water needs to be effectively delivered to the tissues to ensure that the ablation properties of the bone and thermal conductivity remain constant within the limits of the entire ablation process. To counteract the risk of water pooling in the cutting zone, the handpiece 1100 may be equipped with one or more pressurized micro-channels 1801 running parallel with the coherent laser beam 1402 to evacuate excess water 1803 at the cutting interface. The micro-channels 1801 may also promote water circulation to aid in the removal of bone debris 1804 and excess heat 1805 from the heat affected resection site 1802. A water reservoir may be used to feed water to the handpiece via an on/off electromechanical-operated valve. For efficient operation (i.e., fast switching), the valve may be as close as possible to the nozzle of the handpiece.

### Robotically Controlled Laser-Out-Handpiece

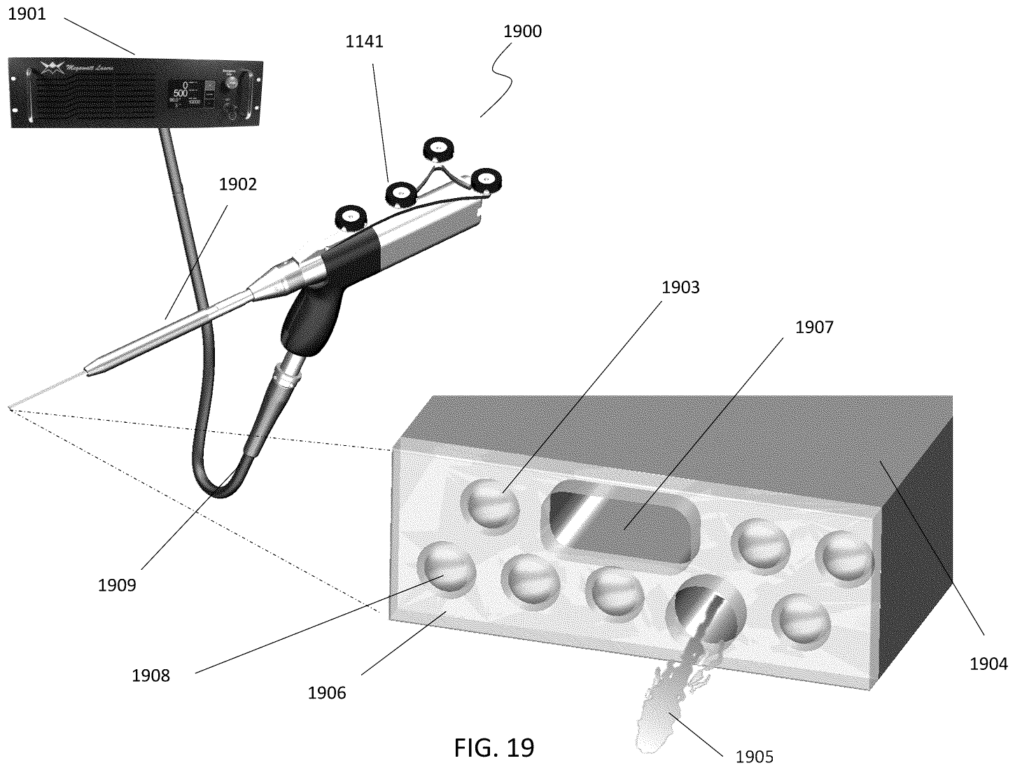

FIG. 19 illustrates a robotically controlled handpiece 1900 connected to an external laser source 1901 (e.g., a 30 W average power Er: YAG laser with 3-micron laser wavelength). The laser can be passed to the handheld device 1900 through a cable harness 1909 comprising one or more fibers. The end effector 1902 of the handpiece 1900 may be configured such that a portion of the end effector 1902 can be plunged into the bone during ablation. In some embodiments, the end effector 1902 includes an array of solid fiber lasers 1903 (e.g., LCPH425-100 solid sapphire 425-micron diameter by 30 to 50 cm in length) operated in a cold ablation regime. Solid sapphire fibers may be preferable to hollow cores given that they are less prone to laser loss/attenuation than hollow core/cladded fibers. In some embodiments, the output face 1908 (i.e., distal end) of each fiber is processed (e.g., similarly to a lens) such that the delivered beam can remain collimated. The proximal multiplexed end can be machined into a hexagon to improve coupling. The overlapping fibers 1903 can be embedded in a polymer substrate 1904 (e.g., PTFE, PET, and/or nylon). In some embodiments, the cutting end of the effector is protected with a disposable external transparent window 1906 (e.g., sapphire) that protects the fiber 1903 lasers from thermal damage and biological contamination enabling them to be re-used. The resultant line-shaped beam with an engineered intensity profile can be created by overlapping several convex-shaped fiber lasers (e.g., 7) to remove bone at rates equivalent to mechanical cutting tools (e.g., 3.5 to 6.9 mm³/s) and depths (e.g., approximately 50 mm) necessary for joint replacement surgery, while preserving the native bone surface. The end effector 1902 can provide internal water cooling 1905 and debris removal capability 1907 (e.g., air suction) improving local thermal conditions.

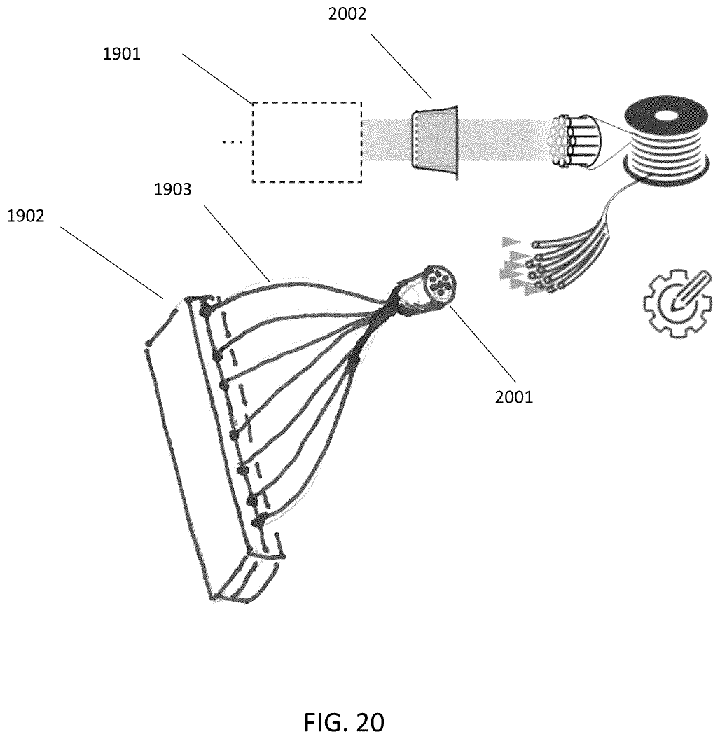

FIG. 20 depicts a ferrule connector 2001 in accordance with an embodiment. The ferrule connector 2001 can be used to connect the multiplexed fibers 1903 embedded in the end effector 1902 with the external laser 1901. Typical settings can include a fiber working length of 50 mm, a repetition rate of less than 10 Hz to avoid prematurely degrading the fibers 1903, a cut distance from the bone surface of 2 mm, a pulse duration of 50 to 500 μs, an energy of 430 mJ per fiber 1903, assuming 7 multiplexed fibers, with a no loss worst case energy of 350 mJ and 3000 mJ per pulse. Sculptured coherent pulse trains can reduce the build-up of heat from pulse to pulse. For example, low-frequency bursts of high-frequency pulses (e.g., 10 pulses of 4 μJ and a 0.5 to 1.7 GHz intraburst repetition rate). Thus, lower laser cutting temperatures can be achieved by decreasing the pulse energy and increasing the number of pulses in each burst and the intra-burst repetition rate. Beam shaping optical elements 2002 may be included in the external laser 1901 or in the handpiece 1900.

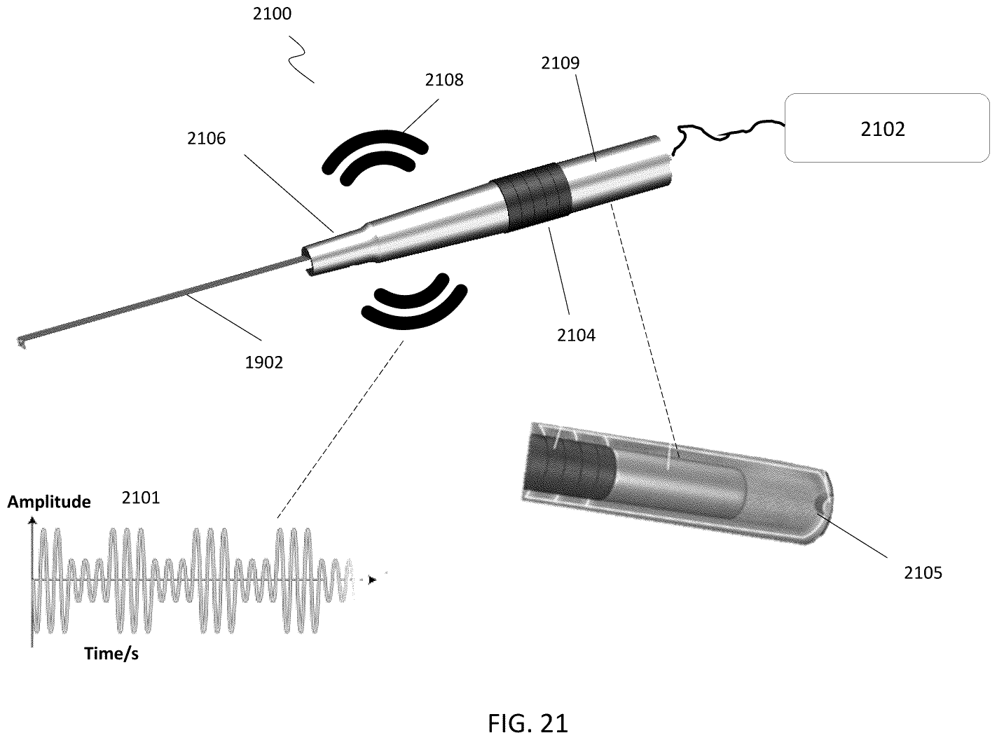

FIG. 21 illustrates a piezoelectric actuation system 2100 which may be included in the handpiece. In some embodiments, the actuator 2100 is operated at between 5 and 10 W. The actuator 2100 can vibrate the end effector 1902, thereby assisting laser cutting in one direction. The vibration frequency 2101 may be programmable and created by driving an electric current from a generator 2102 over piezoceramic rings 2104, which leads to their deformation. The resulting linear movement from deformation of the rings 2104 produces vibration in a transducer 2105 and/or amplifier 2106, which creates the ultrasound output. The output may be enhanced using a resonator 2109. Mechanical waves 2106 are transmitted to the handpiece end effector 1902 where the longitudinal movement produced by the vibrations can increase the cut width of the laser (e.g., from 7 mm to over 20 mm). Bone can be laser ablated by low-frequency “laser-sweeping” (e.g., 20-300 Hz). High-frequency (e.g., 25-30 kHz) mechanical resection, which can be based on vibration, safely removes bone near adjacent soft tissue structures (e.g., tendons, ligaments, nerves, and blood vessels). Horizontal oscillation may prevent the end effector 1902 from jamming as it is plunged into bone eliminating the risk of bone inter-digitation during cutting. Vibrational aid may be included in either laser-in or laser-out embodiments.

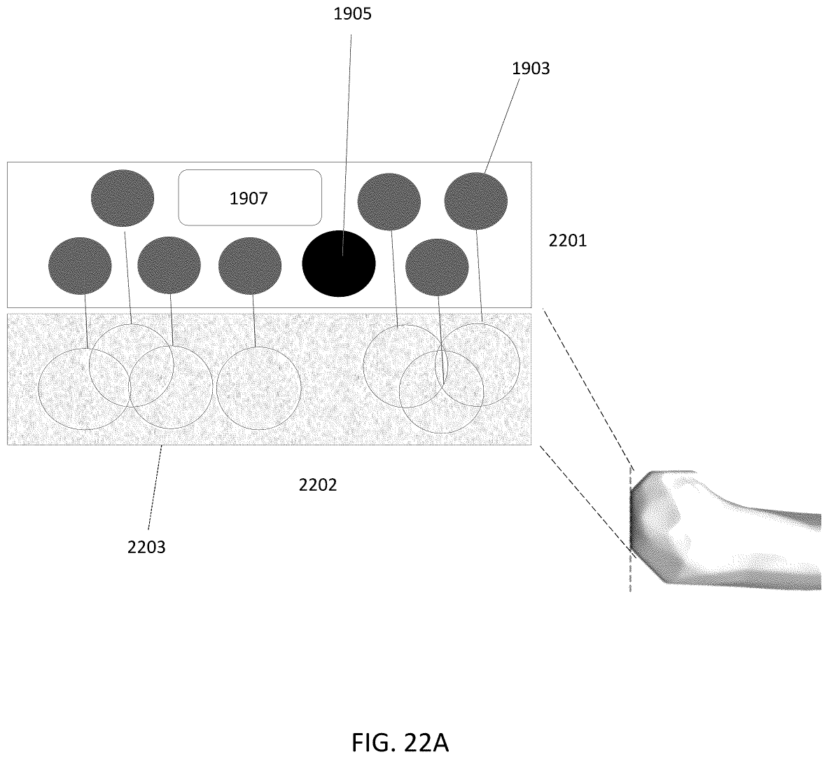

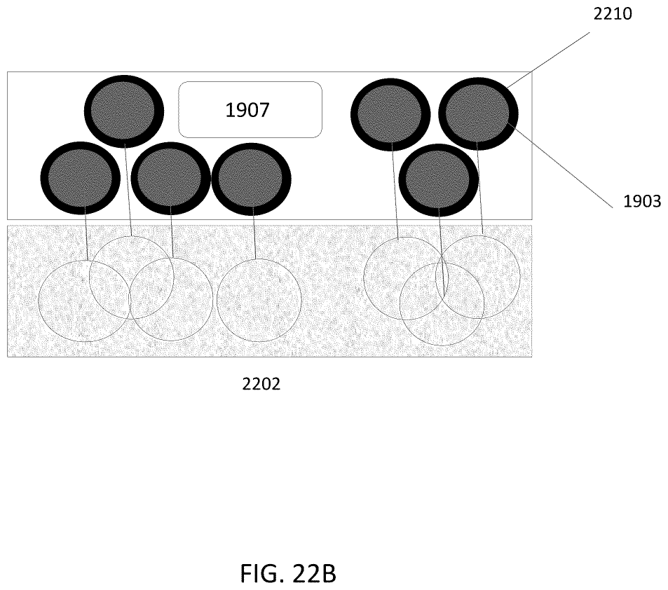

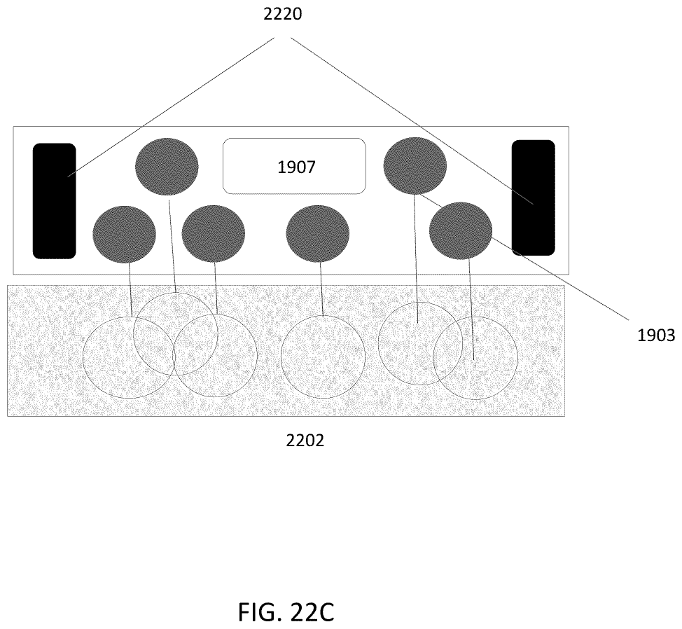

FIGS. 22A-C depict illustrative end views 2201 of the end effector 1902 and the resultant homogenous intensity distribution 2202 in the bone 2203 created by the overlapping fiber lasers 1903. The in-line water coolant 1903 can be delivered separately at the center region, the periphery of the end effector 2220, or around the circumference of the fibers 2210.

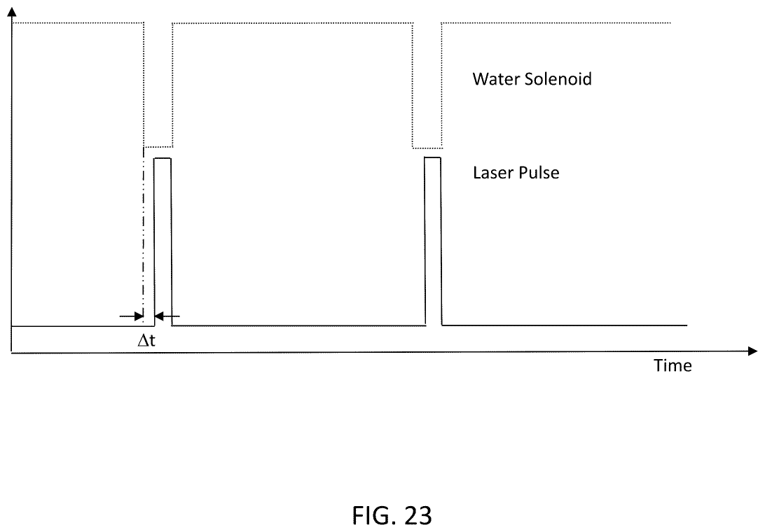

The liquid cooling system can also be improved by controlling water delivery through ON and OFF sequences such that a time delay (Δt) exists between shutting off delivery of the liquid coolant and commencement of laser pulse energy delivery to the site of tissue ablation. FIG. 23 illustrates the timing of the laser pulse relative to the coolant delivery in accordance with an embodiment. The coolant delivery system is timed to deliver coolant to the target area at a time that does not overlap with the laser pulse. The interleaved timing of the laser pulse and coolant has advantages including reduced heat accumulation during laser cutting, removal of the remaining heat in the bone between laser pulses, constantly cleaning the laser path (e.g., removing irrigated water/debris produced during ablation and clearing the focusing lens) on the ablation process, and re-hydrating the adjacent tissue, which prevents carbonization.

Pressurized airflow or an extraction system may be used to improve the application of the laser pulse by blowing debris and excess water away from the cavity and the tip of the handpiece. Pressurized air can be delivered through one or more channels running in parallel with the laser beam in the cutting head. For example, the pressurized air can be delivered at between 0.5 bar and 30 bar, and/or at a laminar flow region of 1 to 2 cm long cooling the ablated area.

The handpiece 1100 may be adapted to sense the depth of the photoablation. In some embodiments, the device further includes real-time optical control supporting laser depth analysis/visualization and involuntary hand movements (e.g., jerking or trembling) that can lead to deviations of the laser beam during cutting. In some embodiments, monitoring can be performed using an IR camera that detects the cutting temperature. In some embodiments, the handpiece 1100 may include a secondary laser (e.g., a class I visible laser such as a helium-neon laser) configured for distance measurement. In some embodiments, the secondary laser may be the same laser used for visual targeting. In some embodiments, a different laser may be used as the secondary laser.

The system may include optoacoustic/photoacoustic imaging. Optoacoustic/Photoacoustic imaging (OAI/PAI) is a medical imaging modality that enables non-invasive visualization of laser-illuminated tissue by the detection of acoustic signals. OAI is used to describe the light-induced sound phenomena that occurs when the excitation light is within the visible and near-infrared portion of the electromagnetic spectrum. Using OAI, laser light may be absorbed by specific components within the tissue, such as hemoglobin or lipids, thereby generating a mechanical wave with a frequency in the ultrasound regime. The mechanical wave can be detected by an ultrasound sensor, or an array of ultrasound sensors, and the signals can be used to form an image using image reconstruction algorithms. The resulting contrast of the image is based upon the distribution of absorbed optical energy within the tissue, which is related to the wavelength of the laser light used and the optical properties of the tissue being scanned. The imaging can also be used to differentiate bone from vital soft tissue structures within the cut plane to avoid any unnecessary tissues resection.

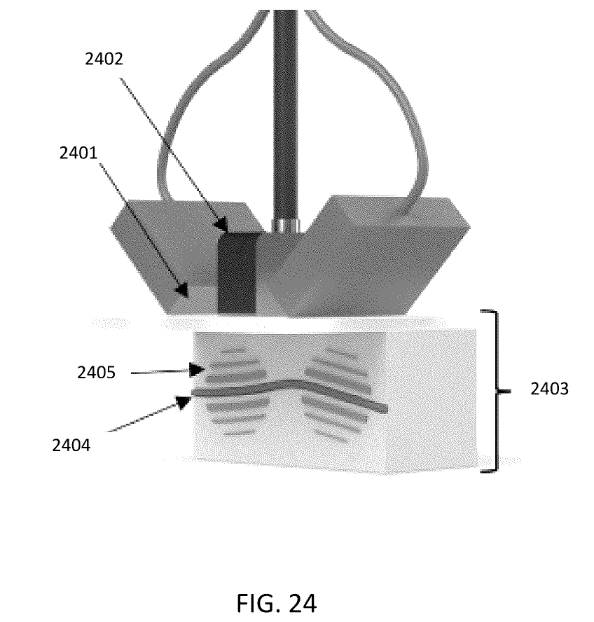

FIG. 24 depicts an Optoacoustic Computed Tomography (OACT) assembly in accordance with an embodiment. OACT can provide improved monitoring to the systems described herein by generating two-dimensional and three-dimensional images. The OACT assembly may use unfocused light 2401, an array of ultrasound transducers 2402, and optical excitation and acoustic 2405 detection to generate images of tissue structures 2404 based upon optical absorption within a tissue sample 2403. The laser may or may not be one of the other lasers described herein (e.g., the treatment laser, targeting laser, or distance laser).

Closed-loop control of the laser bone ablation process can be achieved with a different secondary laser system (e.g., an optical coherence tomography (OCT) system), which can be positioned in an overlapping workspace. The secondary laser system can provide an automatic depth control pathway for bone ablation at a high resolution (e.g., microns) along the z-axis (i.e., the laser beam axis). Depth control can be achieved by combining the working volumes of the OCT system and the Er: YAG ablation laser assuming that the original shape of the bone can be derived from either a 3D OCT scan or some other means (e.g., a statistical shape model). The iterative control cycle may include image processing, path planning, bone ablation of the targeted geometry, and generating a desired cavity depth. Using a mathematical model, the theoretical cavity depth can be calculated for each laser pulse and displayed on the navigation screen. As a result, the material removal can be determined in a volumetric model. Calibration of the OCT system may be required to account for the presence of water vapor or other transparent media in the resection zone.

The number of required laser pulses can be determined based on the thickness of the bone, which may be determined based upon a preoperative CT scan or intra-operative shape model and the known ablation rate per laser pulse (e.g., 400 μm to 500 μm).

In an alternative embodiment, the laser energy emitters (i.e., the laser pump source paired with a laser medium), or a portion thereof, are accommodated inside a remote base unit whereby the laser beam is conveyed to a robotically controlled handheld system via a complex conveying system, such as a long optic fiber or an articulated jointed robotic arm. The system may include a series of optical fibers and mirrors conveying the laser beam from the laser emitter through a cable harness to the handpiece. The handpiece, in such an embodiment, may not require any additional shielding to protect users from electromagnetic radiation emitted from the laser source.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., +10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A robotically controlled laser bone resection device comprising:
a handheld housing comprising:
a treatment laser configured to generate a treatment laser beam,
a laser scanner configured to direct the treatment laser beam,
a water nozzle configured to emit a laminar waterjet, and
an aperture configured to emit the treatment laser beam within the laminar waterjet; and
a tracking marker attached to the handheld housing.

2. The device of claim 1, further comprising:
a handle; and
a gross positioning actuator;
wherein the gross positioning actuator provides two degrees of freedom of movement between the handle and the handheld housing.

3. The device of claim 1, further comprising a fine positioning actuator configured to position the laser scanner.

4. The device of claim 3, further comprising an inertial measurement unit, wherein the fine positioning actuator is configured to adjust the positioning of the treatment laser beam based on detected movement by the inertial measurement unit.

5. The device of claim 1, further comprising a laser emitter within a visible spectrum configured to aid in targeting the treatment laser beam.

6. The device of claim 1, further comprising a laser emitter configured to measure a distance between the device and a target surface.

7. The device of claim 1, further comprising an ultrasound transducer configured to detect acoustic waves generated by optical excitation of a target.

8. The device of claim 1, further comprising optical elements configured to shape the treatment laser beam into a top-hat beam.

9. The device of claim 1, the treatment laser is an erbium yttrium-aluminum-garnet laser.

10. The device of claim 1, further comprising a tip, wherein the treatment laser produces a resection larger than the tip such that the tip can enter the resection.

11. The device of claim 10, wherein the tip is configured to be removeable and disposable.

12. The device of claim 1, further comprising an extraction system comprising a pressurized microcavity running parallel to the treatment laser beam.

13. A surgical laser resection system comprising:
a handheld device comprising:
a treatment laser configured to generate a treatment laser beam,
a laser scanner configured to direct the treatment laser beam,
a water nozzle configured to emit a laminar waterjet,
an aperture configured to emit the treatment laser beam within the laminar waterjet, and
a tracking marker; and
a control unit configured to control the treatment laser and control water flow to the water nozzle;
a tracking system configured to detect a position of the tracking marker;
a power generator electrically coupled to the treatment laser; and
a cable harness interfacing the control unit and the power generator to the handheld device.

14. The system of claim 13, wherein the handheld device further comprises:
a handle; and
a gross positioning actuator,
wherein the gross positioning actuator provides two degrees of freedom of movement between the handle and the aperture.

15. The system of claim 13, further comprising an external control device configured to enable/disable the treatment laser.

16. The system of claim 13, wherein the control unit is configured to automatically disable the treatment laser based on achieving a predetermined resection depth.

17. The system of claim 13, wherein the handheld device further comprises a fine positioning actuator configured to position the laser scanner.

18. The system of claim 17, wherein the handheld device further comprises an inertial measurement unit, wherein the fine positioning actuator is configured to adjust the positioning of the treatment laser beam based on detected movement by the inertial measurement unit.

19. The system of claim 13, wherein the handheld device further comprises a laser emitter within a visible spectrum configured to aid in targeting the treatment laser beam.

20. The system of claim 13, wherein the handheld device further comprises a laser emitter configured to measure a distance between the device and a target surface.
